(19) United States
(12) Patent Application Publication
(10) Pub. No.: US 2025/0109151 A1
(43) Pub. Date: Apr. 3, 2025

(54) SILYL-LIPID N-ACYL L-HOMOSERINE LACTONES AS QUORUM SENSING MOLECULES

(71) Applicant: The Regents of the University of California, Oakland, CA (US)

(72) Inventors: Annaliese Franz, Davis, CA (US); Kelsey Mesa, Oakland, CA (US); Linnea Dolph, Oakland, CA (US)

(21) Appl. No.: 18/832,460

(22) PCT Filed: Jan. 24, 2023
(86) PCT No.: PCT/US2023/011478

§ 371 Date: Jul. 23, 2024

Related U.S. Application Data
(60) Provisional application No. 63/302,537, filed on Jan. 24, 2022.

(51) Int. Cl. C07F 7/10

(52) U.S. Cl. CPC C07F 7/10

(57) ABSTRACT

Disclosed are novel silyl-lipid containing N-acylated homoserine lactone compounds. The compounds may be used in quorum sensing and may have desirable agonist or antagonist activity.

### PRIORITY CLAIM

This application claims priority to U.S. Provisional Application No. 63/302,537, filed on Jan. 24, 2022, the entire contents and disclosures of which are incorporated herein.

### STATEMENT AS TO RIGHTS TO INVENTIONS MADE UNDER FEDERALLY SPONSORED RESEARCH OR DEVELOPMENT

Not applicable.

### FIELD OF THE INVENTION

The present disclosure relates generally to the synthesis and evaluation of silyl-lipid containing N-acylated homoserine lactones. In particular, the present disclosure relates to the synthesis and evaluation of silyl-lipid containing N-acylated homoserine lactones as quorum sensing molecules.

### BACKGROUND OF THE INVENTION

Quorum sensing is a form of intercellular communication that relies on the principle that when a single microorganism releases quorum sensing molecules into the environment, the concentration of such molecules is too low to be detected. However, when sufficient bacteria are present, the quorum sensing molecule concentration reaches a threshold level that allows the microorganism to sense important cell clumps and, in response, activate or inhibit a particular gene. Quorum sensing molecules may therefore also be referred to as autoinducers. Pathogens may use quorum sensing molecules as virulence factors. Many common bacterial pathogens rely on chemical communication systems, such as quorum sensing, to regulate virulence and facilitate host colonization. Because pathogens often require virulence factors to colonize their host and cause disease, targeting the regulation or function of virulence factors represents a potential therapeutic strategy to prevent or treat infection, and has attracted considerable attention as an “anti-virulence” approach.

Quorum sensing coordinates gene expression and physiology of bacterial populations. There are several families of molecule used by microbes to communicate. A universal system relying on type II auto-inducers (AI-2) can be used by all bacteria. Gram-positive bacteria use oligopeptides, although Gram-negative QS display various molecules. Among them, the most studied system is represented by the type I auto-inducers based on N-Acyl-homoserines lactones (AHL).

Pharmacophores, which describe the structure arrangement of essential features of an interaction, are important for early drug design and discovery. They may be used as models to filter and identify molecules with possible agonist activity of quorum sensing and to find possible candidates based on calculations of molecular docking. The International Union of Pure and Applied Chemistry (IUPAC) defines a pharmacophore as an ensemble of steric and electronic features that is necessary to ensure the optimal supramolecular interactions with a specific biological target and to trigger or block its biological response. A pharmacophore model may explain how structurally diverse ligands may bind to a common receptor site. Additionally, pharmacophore models may be used to identify novel ligands that will bind to the same receptor.

Pharmacophore features may include hydrophilic moieties or hydrophobic moieties. Bioisosteres for hydrophilic pharmacophores may come in wide variety with carboxylic acids having around 35 commonly used replacements, described in Lassalas et al., “Structure Property Relationships of Carboxylic Acid Isosteres,” J. Med. Chem. 2016, 59, 3183-3203.

Hydrophobic moieties have been recognized for having comparable importance in protein-ligand complex formation as their hydrophilic counterparts. This is most succinctly demonstrated by the “magic methyl effect,” described by Feng et al. “Late-stage oxidative C(sp³)-H methylation,” Nature 580, 621-627 (2020). The “magic methyl effect” is where the replacement of a hydrogen atom with a methyl group can lead to a 2000 fold increase in potency. These hydrophobic or lipophilic pharmacophores, however, are often limited in their diversity to simple hydrocarbon chains or benzene rings, although there has been some recent development in the use of propellanes.

Silicon is one element useful as a carbon alternative for affecting the design and control of hydrophobic chemical structures. Based on its stability and unique properties, silicon can play an important role in the design of biological probes, pharmaceutical agents, and materials. Silicon is the 2nd most common element on earth, and silanes already have numerous industrial and technology applications in materials and inorganic chemistry. The flexible steric and substitution patterns of silyl groups allow tunable reactivity, stability, and solubility. There is no inherent “element-specific” toxicity or silicon-containing compounds, and silicon has multiple properties relevant for medicinal and clinical applications (G. A. Showell & J. S. Mills, Drug Discov. Today 8, (2003): 551; R. Ramesh & D. S. Reddy, J. Med. Chem. 61, (2018): 3779; W. Baines & R. Tacke, Curr. Opin. Drug Discov. Devel. 6, (2003): 526). The C—Si bond is stable under physiological conditions. Organosilanes can be synthesized using efficient reactions from a variety of commercially available reagents. The silicon atom has a larger covalent radius resulting in the formation of 20% longer Si—X bonds compared to C—X bonds and providing higher conformational flexibility (M. A. Brooks, Silicon in Organic, Organometallic, and Polymer Chemistry, Wiley: New York (2000)). The electropositive nature and bond-polarization of silicon (relative to C, N, and O) contributes to an electron-deficient center in a molecule. Trialkylsilyl groups are more lipophilic than the corresponding trialkylmethyl groups (Log P for trimethylsilyl-benzene=4.7 vs Log P for t-butylbenzene=4.0) (M. A. Brooks, Silicon in Organic, Organometallic, and Polymer Chemistry, Wiley: New York (2000); U. I. Zakai, G. Bikzhanova, D. Staveness, S. Gately & R. West, Appl. Organomet. Chem. 24, (2010): 189). And silicon has the potential to alter metabolic fate to avoid toxic metabolites (T. Johansson, L. Weidolf, F. Popp, R. Tacke & U. Jurva, Drug Metab. Dispos 38, (2010): 73; R. J. Fessednen & R. A. Hartman, J. Med. Chem. 13, (1970): 52; N. A. Meanwell, J. Med. Chem. 54, (1970): 2259).

The incorporation of silicon isosteres into known drug scaffolds is a method to optimize biological activity and reduce toxicity (G. A. Showell & J. S. Mills, Drug Discov. Today 8, (2003): 551; S. Gately & R. West, Drug Dev. Res. 68, (2007): 156; M. Geyer et al., ChemMedChem 10, (2015): 911; A. K. Franz & S. O. Wilson, J. Med. Chem. 56, (2013): 388). Silatecans, silyl analogs of DNA topoisomerase inhibitor camptothecin, have been shown to increase cell penetration, enhance blood stability and improve pharmacokinetics relative to camptothecin (A. A. Gabizon et al., Clin. Cancer Res. 12, (2006): 1913). The enhanced in vivo stability of DB-67 is attributed to the enhanced lipophilicity that reduces in vivo hydrolysis due to increased partitioning in the red blood cells (V. J. Venditto & E. E. Simanek, Mol. Pharm. 7, (2010): 307). Notably, silatecans Karenitecin and DB-67 have advanced to clinical trials (A. A. Gabizon et al., Clin. Cancer Res. 12, (2006): 1913; A. Daud et al., Clin. Cancer Res. 11, (2005): 3009). Several trimetylsilylpyrazole analogs of BIRB-796, a non-peptide inhibitor of p38 mitogen-activated protein (MAP) kinase (J. Regan et al., J. Med. Chem. 45, (2002): 2994; W. N. Sivak et al., Acta Biomater. 4, (2008): 852), have been synthesized as new silicon isosteres for kinase inhibitors (V. J. Venditto & E. E. Simanek, Mol. Pharm. 7, (2010): 307). A sila-analog of BIRB-796 demonstrated enhanced stability to degradation by human liver microsomes and in-vivo data in an LPS-induced model of TNF-α release indicated similar efficacy and also suggested that the silicon analog induces TNF-α suppression more quickly (59% compared to 41% at 30 min) (J. Regan et al., J. Med. Chem. 45, (2002): 2994). Incorporating a silicon-containing amino acid such as γ-(dimethylsila)proline (silaproline, Sip) (M. W. Mutahi, T. Nittoli, L. Guo, & S. M. N. Sieburth, J. Am. Chem. Soc. 124, (2002): 7363; S. Pujals et al., J. Am. Chem. Soc. 128, (2006): 8479), has been shown to increase resistance to proteolytic degradation, increase cellular uptake, and enhance lipophilicity (e.g. Log P is 0.094 for Fmoc-Pro and 1.3 for Fmoc-Sip) (S. Pujals et al., J. Am. Chem. Soc. 128, (2006): 8479). Upon incorporation of silaproline in proline-rich cell-penetrating peptides, the peptides maintained their secondary structure and cellular uptake was enhanced (F. Cavelier et al., J. Am. Chem. Soc. 124, (2002): 2917; F. Cavelier, D. Marchand, J. Martinez & S. Sagan, J. Pepet. Res. 63, (2004): 290). TMS-alanine has been used as a replacement for both phenylalanine and for leucine based on the lipophilicity (R. Fanelli et al., J. Med. Chem. 58, (2015): 7785).

The metabolism of organosilicon molecules is an important consideration where similar metabolic rates and oxidation occurs (R. J. Fessednen &R. A. Hartman, J. Med. Chem. 13, (1970): 52; M. Geyer et al., ChemMedChem 10, (2015): 911), however, the presence of the silicon atom can also alter the metabolic fate to reduce toxicity. For example, sila-haloperidol, a dopamine D2 antagonist, avoids formation of a neurotoxic pyridinium ion metabolite (T. Johansson, L. Weidolf, F. Popp, R. Tacke & U. Jurva, Drug Metab. Dispos 38, (2010): 73; B. Subramanyam, H. Rollema, T. Woolf & N. Castagnoli, Biochem. Biophys. Res. Commun. 16, (1990): 238; R. Tacke et al., ChemMedChem 3, (2008): 152). In general, organosilicon molecules are stable in aqueous and oxygen-rich environments and the metabolism of organosilanes is still generally shown to follow standard pathways.

Despite these examples, there has not been a systematic study for incorporation of silyl groups to strategically alter the structure and conformation of hydrophobic groups for medicinal chemistry. In particular, the inventors are aware of very few reports describing silyl groups incorporated in a fatty acid (D. Kajita et al., Bioorg. Med. Chem. Lett. 25, (2015): 3350; U.S. Pat. No. 8,895,769). In view of these limitations in the available knowledge related to silyl lipids, as well as the aforementioned shortcomings in applying existing AHLs for quorum sensing, there are currently unmet needs for additional approaches that can provide improved compounds with enhanced therapeutic efficacy and selectivity. The materials and methods disclosed herein address these and other needs.

### SUMMARY OF THE INVENTION

In some embodiments, the present disclosure is directed to a compound having the formula:

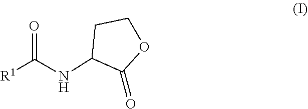

wherein R¹ has the formula:

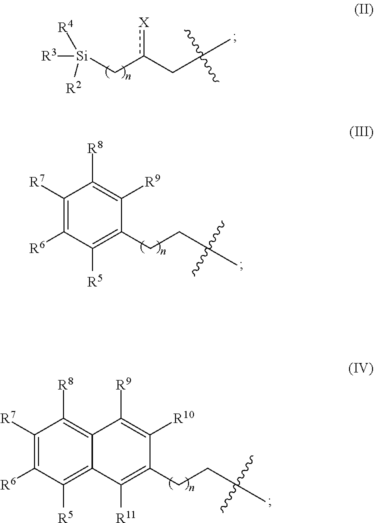

wherein R²; R³, and R⁴ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group;
wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group, with the proviso that at least one of R⁵, R⁶, R⁷, R⁸, R⁹, R¹⁰, and R¹¹ comprises Si;
wherein X is hydrogen, or oxygen,
wherein the subscript n is an integer from 0 to 9;
or a pharmaceutically acceptable salt, solvate, or isomer thereof.

In some embodiments, R¹ is Formula (II). In some embodiments, R¹ is Formula (II) and wherein R², R³, and R⁴ are independently selected from a C₁₋₁₀ alkyl group. In some embodiments, R¹ is Formula (II) and wherein R², R³, and R⁴ are independently selected from a C₁₋₅ alkyl group. In some embodiments, R¹ is Formula (II) and wherein at least two of R², R³, and R⁴ are identical. In some embodiments, R¹ is Formula (II) and wherein R², R³, and R⁴ are identical and are selected from a C₁₋₁₀ alkyl group. In some embodiments, least one of R², R³, and R⁴ is a phenyl group or an alkyl phenyl group.

In some embodiments, R¹ is Formula (III). In some embodiments, two of R⁵, R⁶, R⁷, R⁸, and R⁹ are hydrogen. In some embodiments, three of R⁵, R⁶, R⁷, R⁸, and R⁹ are hydrogen. In some embodiments, four of R⁵, R⁶, R⁷, R⁸, and R⁹ are hydrogen.

In some embodiments, at least one of R⁵, R⁶, R⁷, R⁸, and R⁹ has the formula:

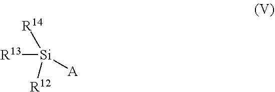

wherein A is at least one of R⁵, R⁶, R⁷, R⁸, and R⁹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

In some embodiments, at least one of R¹², R¹³, and R¹⁴ is a phenyl group or an alkyl phenyl group. In some embodiments, at least one of R¹², R¹³, and R¹⁴ is an alkyl group. In some embodiments, R¹ is Formula (IV). In some embodiments, two of R⁵, R⁶, R⁷, R⁸, R⁹, R¹⁰ and R¹¹ are hydrogen. In some embodiments, three of R⁵, R⁶, R⁷, R⁸, R⁹, R¹⁰ and R¹¹ are hydrogen. In some embodiments, four of R⁵, R⁶, R⁷, R⁸, R⁹, R¹⁰ and R¹¹ are hydrogen. In some embodiments, five of R⁵, R⁶, R⁷, R⁸, R⁹, R¹⁰ and R¹¹ are hydrogen. In some embodiments, six of R⁵, R⁶, R⁷, R⁸, R⁹, R¹⁰ and R¹¹ are hydrogen.

In some embodiments, at least one of R⁵, R⁶, R⁷, R⁸, R⁹, R¹⁰ and R¹¹ has the formula:

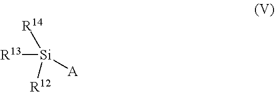

wherein A is at least one of R⁵, R⁶, R⁷, R⁸, R⁹, R¹⁰ and R¹¹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

In some embodiments, at least one of R¹², R¹³, and R¹⁴ is a phenyl group. In some embodiments, at least one of R¹², R¹³, and R¹⁴ is an alkyl group.

In some aspects, in any of the above embodiments, X is O. In other aspects, in any of the above embodiments, X is H.

In some embodiments, the compound is (S)—N-(2-oxotetrahydrofuran-3-yl)-5-(triethylsilyl)pentanamide. In some embodiments, the compound is (S)-5-(dimethyl(octyl)silyl)-N-(2-oxotetrahydrofuran-3-yl)pentanamide. In some embodiments, the compound is (S)-2-(4-(butyldimethylsilyl)phenyl)-N-(2-oxotetrahydrofuran-3-yl)acetamide. In some embodiments, the compound is (S)-3-oxo-N-(2-oxotetrahydrofuran-3-yl)-7-(triethylsilyl)heptanamide. In some embodiments, the compound is (S)-2-(4-(dimethyl(phenyl)silyl)phenyl)-N-(2-oxotetrahydrofuran-3-yl)acetamide. In some embodiments, the compound is (S)—N-(2-oxocyclopentyl)-2-(4-(triethylsilyl)phenyl)acetamide. In some embodiments, the compound is (S)-5-(dimethyl(phenyl)silyl)-N-(2-oxotetrahydrofuran-3-yl)pentanamide. In some embodiments, the compound is (S)-5-(butyldimethylsilyl)-N-(2-oxotetrahydrofuran-3-yl)pentanamide. In some embodiments, the compound is (S)—N-(2-oxotetrahydrofuran-3-yl)-5-(tributylsilyl)pentanamide. In some embodiments, the compound is(S)—N-(2-oxotetrahydrofuran-3-yl)-5-(1,1,3,3,3-pentamethyldisiloxaneyl)pentanamide. In some embodiments, the compound is (S)-7-(dimethyl(phenyl)silyl)-N-(2-oxotetrahydrofuran-3-yl)heptanamide. In some embodiments, the compound is (S)-7-(butyldimethylsilyl)-N-(2-oxotetrahydrofuran-3-yl)heptanamide. In some embodiments, the compound is (S)—N-(2-oxotetrahydrofuran-3-yl)-7-(triethylsilyl)heptanamide. In some embodiments, the compound is (S)-7-(dimethyl(phenyl)silyl)-3-oxo-N-(2-oxotetrahydrofuran-3-yl)heptanamide. In some embodiments, the compound is (S)-7-(butyldimethylsilyl)-3-oxo-N-(2-oxotetrahydrofuran-3-yl)heptanamide. In some embodiments, the compound is (S)-2-(4-(dimethyl(octyl)silyl)phenyl)-N-(2-oxotetrahydrofuran-3-yl)acetamide. In some embodiments, the compound is (S)-7-(dimethyl(octyl)silyl)-N-(2-oxotetrahydrofuran-3-yl)heptanamide.

In further embodiments, any of the above disclosed compounds may be used for quorum sensing.

### BRIEF DESCRIPTION OF THE DRAWINGS

FIG. 1 illustrates assay results in accordance with embodiments of the invention.

FIG. 2 illustrates assay results in accordance with embodiments of the invention.

FIG. 3 illustrates assay results in accordance with embodiments of the invention.

FIG. 4 illustrates assay results in accordance with embodiments of the invention.

FIG. 5 illustrates assay results in accordance with embodiments of the invention.

FIG. 6 illustrates assay results in accordance with embodiments of the invention.

FIG. 7 illustrates assay results in accordance with embodiments of the invention.

FIG. 8 illustrates assay results in accordance with embodiments of the invention.

FIG. 9 illustrates assay results in accordance with embodiments of the invention.

FIG. 10 illustrates assay results in accordance with embodiments of the invention.

FIG. 11 illustrates assay results in accordance with embodiments of the invention.

FIG. 12 illustrates assay results in accordance with embodiments of the invention.

FIG. 13 illustrates assay results in accordance with embodiments of the invention.

FIG. 14 illustrates assay results in accordance with embodiments of the invention.

FIG. 15 illustrates assay results in accordance with embodiments of the invention.

FIG. 16 illustrates assay results in accordance with embodiments of the invention.

FIG. 17 illustrates assay results in accordance with embodiments of the invention.

FIG. 18 illustrates assay results in accordance with embodiments of the invention.

FIG. 19 illustrates a reaction scheme in accordance with embodiments of the invention.

### DETAILED DESCRIPTION

### Introduction

The present disclosure describes novel silyl-lipid containing N-acylated Homoserine Lactones (AHL) compounds, their synthesis, and their uses. For example, these novel AHL compounds may be used as quorum sensing molecules, such as for biofilms. The AHL compound may have a structure according to Formula (I):

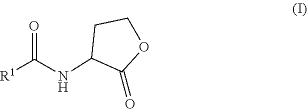

wherein R¹ has the formula:

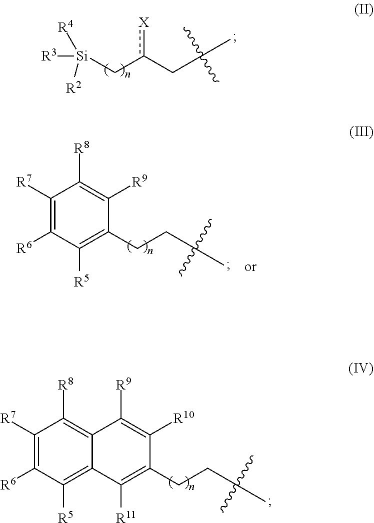

wherein R²; R³, and R⁴ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group;
wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group, with the proviso that at least one of R⁵, R⁶, R⁷, R⁸, R⁹, R¹⁰, and R¹¹ comprises Si;
wherein X is hydrogen, or oxygen,
wherein the subscript n is an integer from 0 to 9;
   or a pharmaceutically acceptable salt, solvate, or isomer thereof.

The compounds disclosed herein may be used in quorum sensing. In some aspects, the compounds may have biological activity that matches the activity of known native ligands or even exceeds it, e.g., by at least 1%, at least 5%, at least 10%, at least 15%, at least 20%, or at least 25%, and in some aspects, up to 100%. In some aspects, the compounds may have an agonist activity (measured as a maximum activation %), such as for LasR activation in E. coli, of greater than 25%, greater than 50%, greater than 75%, greater than 85%, greater than 90%, greater than 95%, greater than 100%, or greater than 105%, and in some aspects, up to 150%. In some aspects, the compounds may have an antagonist activity (measured as a maximum activation %), such as for LasR activation in E. coli, of greater than 25%, greater than 50%, greater than 75%, greater than 85%, greater than 90%, greater than 95%, greater than 100%, or greater than 105%, and in some aspects, up to 150%.

Bioisostere replacements are also common to modulate pharmacokinetic properties including administration, distribution, metabolism, excretion, and toxicity (ADMET), and also help create novel structures to optimize biological activity and reduce toxicity to increase the therapeutic potential of a drug or repurpose the drug. Based on their stability and unique properties, organosilicon molecules offer an opportunity to create bioisosteres for biological probes and pharmaceutical agents. Literature specifically supports that silicon-carbon bioisosteres can have improved PK/PD properties. For example, oxazolidinone antibiotics analogs demonstrated that silicon incorporation increases brain to plasma ratio, credited to the increased lipophilicity and improved plasma and microsomal stability. Notably, there is no inherent “element-specific” toxicity of silicon-containing compounds and the Si—C bond is also stable under physiological conditions. The presence of silicon has also been demonstrated to alter the metabolic pathways and, in some cases, reduce toxicity (e.g., sila-haloperidol). As described further herein, the novel compounds containing Si may be used in quorum sensing and help to expand hydrophobic options for quorum sensing. Further details are described herein.

### Definitions

The abbreviations used herein have their conventional meaning within the chemical and biological arts. Description of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions. Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “salt” refers to acid or base salts of the compounds used in the methods of the present disclosure. A “pharmaceutically acceptable salt” is one that is compatible with other ingredients of a formulation composition containing the compound, and that is not deleterious to a recipient thereof. It is thus understood that the pharmaceutically acceptable salts are non-toxic. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present disclosure are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

As used herein, the term “solvate” refers to a compound that is complexed to at least one solvent molecule. The compounds of the present disclosure may be complexed with from 1 to 10 solvent molecules. In some embodiments, the solvent is water and the solvate is a hydrate.

As used herein, the term “isomers” refers to compounds having the same number and kind of atoms, and hence the same molecular weight, but differing in respect to the structural arrangement or configuration of the atoms. Isomers thus include compounds having different arrangements of the same formula of atoms in a molecule possessing one or more asymmetric carbon atoms or double bonds. Isomers may include racemates, enantiomers, diastereomers, geometric isomers, and individual isomers.

As used herein, the term “alkyl” refers to a straight or branched, saturated, aliphatic radical having the number of carbon atoms indicated. Alkyl may include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl may also refer to alkyl groups having up to 20 carbons atoms, such as, but not limited to heptyl, octyl, nonyl, decyl, etc.

As used herein, the term “alkenyl” refers to a straight chain or branched hydrocarbon having at least 2 carbon atoms and at least one double bond. Alkenyl may include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups may have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, and 1,3,5-hexatrienyl. Alkenyl groups can be substituted or unsubstituted.

As used herein, the term “alkoxy” refers to a substituted alkyl group, as defined above, having an oxygen atom that connects the alkyl group to the point of attachment: alkyl-O—. As for the (unsubstituted) alkyl group, alkoxy groups may have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

As used herein, the term “hydroxy” refers to a group consisting of hydrogen bonded to oxygen: —OH.

As used herein, the term “hydroxyalkyl” refers to a substituted alkyl group, as defined above, where at least one of the hydrogen atoms is replaced with a hydroxy group. As for the (unsubstituted) alkyl group, hydroxyalkyl groups may have any suitable number of carbon atoms, such as C₁₋₆. Exemplary hydroxyaryl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

As used herein, the term “aryl” refers to an aromatic ring system having any suitable number of ring atoms and any suitable number of rings. Aryl groups may include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups may be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups, such as phenyl, naphthyl and biphenyl, have from 6 to 12 ring members.

As used herein, the term “alkyl-aryl” refers to a radical having an alkyl component and an aryl component, each as defined above, where the alkyl component links the aryl component to the point of attachment. The alkyl component is as defined above, except that the alkyl component is at least divalent, i.e., is an alkylene, to link to the aryl component and to the point of attachment. The alkyl component can include any number of carbons, such as C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆, and C₅₋₆. Examples of the alkyl-alkoxy group include, but are not limited to, 2-ethoxy-ethyl and methoxymethyl. Exemplary alkyl-aryl groups include, but are not limited to, phenylmethyl, phenylethyl (where the phenyl is in the 1- or 2-position), phenylpropyl (where the phenyl is in the 1-, 2- or 3-position), phenylbutyl (where the phenyl is in the 1-, 2-, 3- or 4-position), phenylpentyl (where the phenyl is in the 1-, 2-, 3-, 4- or 5-position), phenylhexyl (where the phenyl is in the 1-, 2-, 3-, 4-, 5- or 6-position), 1,2-diphenylethyl, and the like.

As used herein, the term “halogen” refers to fluorine, chlorine, bromine, and iodine.

As used herein, the term “haloalkyl” refers to a substituted alkyl, as defined above, where at least one of the hydrogen atoms is replaced with a halogen atom. As for the (unsubstituted) alkyl group, haloalkyl groups may have any suitable number of carbon atoms, such as C₁₋₆. For example, haloalkyl includes trifluoromethyl, fluoromethyl, etc. In some instances, the term “perfluoro” may be used to define a compound or radical where all the hydrogens are replaced with fluorine. For example, perfluoromethane includes 1,1,1-trifluoromethyl.

As used herein, the term “nitro” refers to a group consisting of two oxygen atoms bonded to nitrogen: —NO₂.

As used herein, the term “cyano” refers to a group consisting of nitrogen triple-bonded to carbon: —C≡N.

As used herein, the term “silyl” refers to a group having at least one carbon atom (alkyl group(s)) bonded to silicone: —Si—C.

As used herein, the term “siloxy” refers to a group having a silicone bonded to oxygen: —Si—O.

As used herein, the term “agonist” refers to a substance that has an affinity for the active site of a receptor and thereby preferentially stabilizes the active state of the receptor, or a substance that produces activation of receptors and enhances signaling by those receptors.

As used herein, the term “composition” refers to a product comprising the specified ingredients in the specified amounts, as well as any product, which results, directly or indirectly, from combination of the specified ingredients in the specified amounts. A “pharmaceutically acceptable composition” is one in which each ingredient, e.g., a carrier, diluent or excipient, is compatible with the other ingredients of a formulation composition and not deleterious to the recipient thereof.

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the singular forms “a”, “an” and “the” include plural referents unless the content clearly dictates otherwise. Thus, for example, reference to “a pharmaceutically acceptable carrier” optionally includes a combination of two or more pharmaceutically acceptable carriers, and the like.

### Compounds

The novel silyl-containing amphiphilic lactone compounds described herein may have the structure of Formula (I):

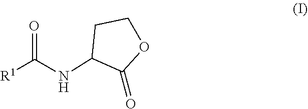

wherein R¹ has the formula:

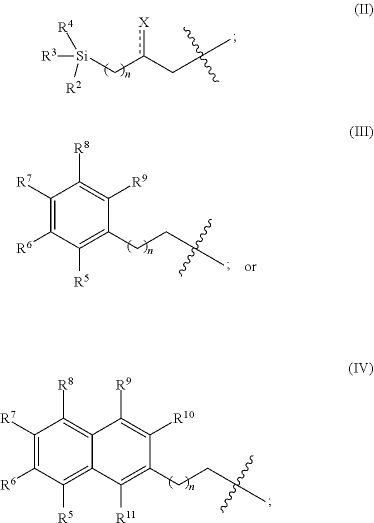

wherein R²; R³, and R⁴ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group;
wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group, with the proviso that at least one of R⁵, R⁶, R⁷, R⁸, R⁹, R¹⁰, and R¹¹ comprises Si;
wherein X is hydrogen, or oxygen,
wherein the subscript n is an integer from 0 to 9;
   or a pharmaceutically acceptable salt, solvate, or isomer thereof.

In some embodiments, R¹ of formula I is

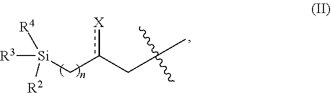

wherein R²; R³, and R⁴ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

In some embodiments, R² is hydrogen. In some embodiments, R² is C₁₋₁₀ alkyl. In some embodiments, R² is methyl. In some embodiments, R² is ethyl. In some embodiments, R² is propyl. In some embodiments, R² is isopropyl. In some embodiments, R² is butyl. In some embodiments, R² is isobutyl. In some embodiments, R² is sec-butyl. In some embodiments, R² is tert-butyl. In some embodiments, R² is pentyl. In some embodiments, R² is isopentyl. In some embodiments, R² is 2-methylbutyl. In some embodiments, R² is pentan-2-yl. In some embodiments, R² is 3-methylbutan-2-yl. In some embodiments, R² is pentan-3-yl. In some embodiments, R² is neopentyl. In some embodiments, R² is tert-pentyl. In some embodiments, R² is hexyl. In some embodiments, R² is 4-methylpentyl. In some embodiments, R² is 3-methylpentyl. In some embodiments, R² is 2-methylpentyl. In some embodiments, R² is hexan-2-yl. In some embodiments, R² is 2,3-dimethylbutyl. In some embodiments, R² is 4-methylpentan-2-yl. In some embodiments, R² is 3-methylpentan-2-yl. In some embodiments, R² is 2-ethylbutyl. In some embodiments, R² is hexan-3-yl. In some embodiments, R² is 3,3-dimethylbutyl. In some embodiments, R² is 2,2-dimethylbutyl. In some embodiments, R² is 2-methylpentan-2-yl. In some embodiments, R² is C₂₋₆ alkenyl. In some embodiments, R² is phenyl. In some embodiments, R² is alkyl phenyl. In some embodiments, R² is a C₁₋₆ alkyl phenyl group. In some embodiments, R² is a C₂ alkyl phenyl group. In some embodiments, R² is a naphthyl group. In some embodiments, R² is a C₁₋₆ alkyl naphthyl group. In some embodiments, R² is a C₂ alkyl naphthyl group. In some embodiments, R² is a siloxy group. In some embodiments, R² is a silyl group.

In some embodiments, R³ is hydrogen. In some embodiments, R³ is C₁₋₁₀ alkyl. In some embodiments, R³ is methyl. In some embodiments, R³ is ethyl. In some embodiments, R³ is propyl. In some embodiments, R³ is isopropyl. In some embodiments, R³ is butyl. In some embodiments, R³ is isobutyl. In some embodiments, R³ is sec-butyl. In some embodiments, R³ is tert-butyl. In some embodiments, R³ is pentyl. In some embodiments, R³ is isopentyl. In some embodiments, R³ is 2-methylbutyl. In some embodiments, R³ is pentan-2-yl. In some embodiments, R³ is 3-methylbutan-2-yl. In some embodiments, R³ is pentan-3-yl. In some embodiments, R³ is neopentyl. In some embodiments, R³ is tert-pentyl. In some embodiments, R³ is hexyl. In some embodiments, R³ is 4-methylpentyl. In some embodiments, R³ is 3-methylpentyl. In some embodiments, R³ is 2-methylpentyl. In some embodiments, R³ is hexan-2-yl. In some embodiments, R³ is 2,3-dimethylbutyl. In some embodiments, R³ is 4-methylpentan-2-yl. In some embodiments, R³ is 3-methylpentan-2-yl. In some embodiments, R³ is 2-ethylbutyl. In some embodiments, R³ is hexan-3-yl. In some embodiments, R³ is 3,3-dimethylbutyl. In some embodiments, R³ is 2,2-dimethylbutyl. In some embodiments, R³ is 2-methylpentan-2-yl. In some embodiments, R³ is C₂₋₆ alkenyl. In some embodiments, R³ is phenyl. In some embodiments, R³ is alkyl phenyl. In some embodiments, R³ is a C₁₋₆ alkyl phenyl group. In some embodiments, R³ is a C₃ alkyl phenyl group. In some embodiments, R³ is a naphthyl group. In some embodiments, R³ is a C₁₋₆ alkyl naphthyl group. In some embodiments, R³ is a C₂ alkyl naphthyl group. In some embodiments, R³ is a siloxy group. In some embodiments, R³ is a silyl group.

In some embodiments, R⁴ is hydrogen. In some embodiments, R⁴ is C₁₋₁₀ alkyl. In some embodiments, R⁴ is methyl. In some embodiments, R⁴ is ethyl. In some embodiments, R⁴ is propyl. In some embodiments, R⁴ is isopropyl. In some embodiments, R⁴ is butyl. In some embodiments, R⁴ is isobutyl. In some embodiments, R⁴ is sec-butyl. In some embodiments, R⁴ is tert-butyl. In some embodiments, R⁴ is pentyl. In some embodiments, R⁴ is isopentyl. In some embodiments, R⁴ is 2-methylbutyl. In some embodiments, R⁴ is pentan-2-yl. In some embodiments, R⁴ is 3-methylbutan-2-yl. In some embodiments, R⁴ is pentan-3-yl. In some embodiments, R⁴ is neopentyl. In some embodiments, R⁴ is tert-pentyl. In some embodiments, R⁴ is hexyl. In some embodiments, R⁴ is 4-methylpentyl. In some embodiments, R⁴ is 3-methylpentyl. In some embodiments, R⁴ is 2-methylpentyl. In some embodiments, R⁴ is hexan-2-yl. In some embodiments, R⁴ is 2,3-dimethylbutyl. In some embodiments, R⁴ is 4-methylpentan-2-yl. In some embodiments, R⁴ is 3-methylpentan-2-yl. In some embodiments, R⁴ is 2-ethylbutyl. In some embodiments, R⁴ is hexan-3-yl. In some embodiments, R⁴ is 3,3-dimethylbutyl. In some embodiments, R⁴ is 2,2-dimethylbutyl. In some embodiments, R⁴ is 2-methylpentan-2-yl. In some embodiments, R⁴ is C₂₋₆ alkenyl. In some embodiments, R⁴ is phenyl. In some embodiments, R⁴ is alkyl phenyl. In some embodiments, R⁴ is a C₁₋₆ alkyl phenyl group. In some embodiments, R⁴ is a C₂ alkyl phenyl group. In some embodiments, R⁴ is a naphthyl group. In some embodiments, R⁴ is a C₁₋₆ alkyl naphthyl group. In some embodiments, R⁴ is a C₂ alkyl naphthyl group. In some embodiments, R⁴ is a siloxy group. In some embodiments, R⁴ is a silyl group.

In some embodiments, R², R³, and R⁴ are independently selected from a C₁₋₁₀ alkyl group. In some embodiments, R², R³, and R⁴ are independently selected from a C₁₋₅ alkyl group. In some embodiments, at least two of R², R³, and R⁴ are identical. In some embodiments, R², R³, and R⁴ are identical and are selected from a C₁₋₁₀ alkyl group. In some embodiments, at least one of R², R³, and R⁴ is a phenyl group, an alkyl phenyl group (such as CH₂CH₂ phenyl).

In some embodiments, R¹ of formula I is

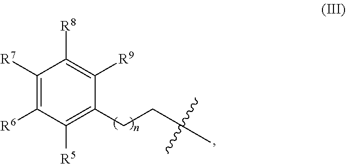

wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group, with the proviso that at least one of R⁵, R⁶, R⁷, R⁸, R⁹, R¹⁰, and R¹¹ comprises Si. In some embodiments, R⁵ is hydrogen. In some embodiments, R⁵ is C₁₋₁₀ alkyl. In some embodiments, R⁵ is methyl. In some embodiments, R⁵ is ethyl. In some embodiments, R⁵ is propyl. In some embodiments, R⁵ is isopropyl. In some embodiments, R⁵ is butyl. In some embodiments, R⁵ is isobutyl. In some embodiments, R⁵ is sec-butyl. In some embodiments, R⁵ is tert-butyl. In some embodiments, R⁵ is pentyl. In some embodiments, R⁵ is isopentyl. In some embodiments, R⁵ is 2-methylbutyl. In some embodiments, R⁵ is pentan-2-yl. In some embodiments, R⁵ is 3-methylbutan-2-yl. In some embodiments, R⁵ is pentan-3-yl. In some embodiments, R⁵ is neopentyl. In some embodiments, R⁵ is tert-pentyl. In some embodiments, R⁵ is hexyl. In some embodiments, R⁵ is 4-methylpentyl. In some embodiments, R⁵ is 3-methylpentyl. In some embodiments, R⁵ is 2-methylpentyl. In some embodiments, R⁵ is hexan-2-yl. In some embodiments, R⁵ is 2,3-dimethylbutyl. In some embodiments, R⁵ is 4-methylpentan-2-yl. In some embodiments, R⁵ is 3-methylpentan-2-yl. In some embodiments, R⁵ is 2-ethylbutyl. In some embodiments, R⁵ is hexan-3-yl. In some embodiments, R⁵ is 3,3-dimethylbutyl. In some embodiments, R⁵ is 2,2-dimethylbutyl. In some embodiments, R⁵ is 2-methylpentan-2-yl. In some embodiments, R⁵ is C₂₋₆ alkenyl. In some embodiments, R⁵ is phenyl. In some embodiments, R⁵ is alkyl phenyl. In some embodiments, R⁵ is a C₁₋₆ alkyl phenyl group. In some embodiments, R⁵ is a C₂ alkyl phenyl group. In some embodiments, R⁵ is a naphthyl group. In some embodiments, R⁵ is a C₁₋₆ alkyl naphthyl group. In some embodiments, R⁵ is a C₂ alkyl naphthyl group. In some embodiments, R⁵ is a siloxy group. In some embodiments, R⁵ is a silyl group.

In some embodiments, R⁶ is hydrogen. In some embodiments, R⁶ is C₁₋₁₀ alkyl. In some embodiments, R⁶ is methyl. In some embodiments, R⁶ is ethyl. In some embodiments, R⁶ is propyl. In some embodiments, R⁶ is isopropyl. In some embodiments, R⁶ is butyl. In some embodiments, R⁶ is isobutyl. In some embodiments, R⁶ is sec-butyl. In some embodiments, R⁶ is tert-butyl. In some embodiments, R⁶ is pentyl. In some embodiments, R⁶ is isopentyl. In some embodiments, R⁶ is 2-methylbutyl. In some embodiments, R⁶ is pentan-2-yl. In some embodiments, R⁶ is 3-methylbutan-2-yl. In some embodiments, R⁶ is pentan-3-yl. In some embodiments, R⁶ is neopentyl. In some embodiments, R⁶ is tert-pentyl. In some embodiments, R⁶ is hexyl. In some embodiments, R⁶ is 4-methylpentyl. In some embodiments, R⁶ is 3-methylpentyl. In some embodiments, R⁶ is 2-methylpentyl. In some embodiments, R⁶ is hexan-2-yl. In some embodiments, R⁶ is 2,3-dimethylbutyl. In some embodiments, R⁶ is 4-methylpentan-2-yl. In some embodiments, R⁶ is 3-methylpentan-2-yl. In some embodiments, R⁶ is 2-ethylbutyl. In some embodiments, R⁶ is hexan-3-yl. In some embodiments, R⁶ is 3,3-dimethylbutyl. In some embodiments, R⁶ is 2,2-dimethylbutyl. In some embodiments, R⁶ is 2-methylpentan-2-yl. In some embodiments, R⁶ is C₂₋₆ alkenyl. In some embodiments, R⁶ is phenyl. In some embodiments, R⁶ is alkyl phenyl. In some embodiments, R⁶ is a C₁₋₆ alkyl phenyl group. In some embodiments, R⁶ is a C₂ alkyl phenyl group. In some embodiments, R⁶ is a naphthyl group. In some embodiments, R⁶ is a C₁₋₆ alkyl naphthyl group. In some embodiments, R⁶ is a C₂ alkyl naphthyl group. In some embodiments, R⁶ is a siloxy group. In some embodiments, R⁶ is a silyl group.

In some embodiments, R⁷ is hydrogen. In some embodiments, R⁷ is C₁₋₁₀ alkyl. In some embodiments, R⁷ is methyl. In some embodiments, R⁷ is ethyl. In some embodiments, R⁷ is propyl. In some embodiments, R⁷ is isopropyl. In some embodiments, R⁷ is butyl. In some embodiments, R⁷ is isobutyl. In some embodiments, R⁷ is sec-butyl. In some embodiments, R⁷ is tert-butyl. In some embodiments, R⁷ is pentyl. In some embodiments, R⁷ is isopentyl. In some embodiments, R⁷ is 2-methylbutyl. In some embodiments, R⁷ is pentan-2-yl. In some embodiments, R⁷ is 3-methylbutan-2-yl. In some embodiments, R⁷ is pentan-3-yl. In some embodiments, R⁷ is neopentyl. In some embodiments, R⁷ is tert-pentyl. In some embodiments, R⁷ is hexyl. In some embodiments, R⁷ is 4-methylpentyl. In some embodiments, R⁷ is 3-methylpentyl. In some embodiments, R⁷ is 2-methylpentyl. In some embodiments, R⁷ is hexan-2-yl. In some embodiments, R⁷ is 2,3-dimethylbutyl. In some embodiments, R⁷ is 4-methylpentan-2-yl. In some embodiments, R⁷ is 3-methylpentan-2-yl. In some embodiments, R⁷ is 2-ethylbutyl. In some embodiments, R⁷ is hexan-3-yl. In some embodiments, R⁷ is 3,3-dimethylbutyl. In some embodiments, R⁷ is 2,2-dimethylbutyl. In some embodiments, R⁷ is 2-methylpentan-2-yl. In some embodiments, R⁷ is C₂₋₆ alkenyl. In some embodiments, R⁷ is phenyl. In some embodiments, R⁷ is alkyl phenyl. In some embodiments, R⁷ is a C₁₋₆ alkyl phenyl group. In some embodiments, R⁷ is a C₂ alkyl phenyl group. In some embodiments, R⁷ is a naphthyl group. In some embodiments, R⁷ is a C₁₋₆ alkyl naphthyl group. In some embodiments, R⁷ is a C₂ alkyl naphthyl group. In some embodiments, R⁷ is a siloxy group. In some embodiments, R⁷ is a silyl group.

In some embodiments, R⁸ is hydrogen. In some embodiments, R⁸ is C₁₋₁₀ alkyl. In some embodiments, R⁸ is methyl. In some embodiments, R⁸ is ethyl. In some embodiments, R⁸ is propyl. In some embodiments, R⁸ is isopropyl. In some embodiments, R⁸ is butyl. In some embodiments, R⁸ is isobutyl. In some embodiments, R⁸ is sec-butyl. In some embodiments, R⁸ is tert-butyl. In some embodiments, R⁸ is pentyl. In some embodiments, R⁸ is isopentyl. In some embodiments, R⁸ is 2-methylbutyl. In some embodiments, R⁸ is pentan-2-yl. In some embodiments, R⁸ is 3-methylbutan-2-yl. In some embodiments, R⁸ is pentan-3-yl. In some embodiments, R⁸ is neopentyl. In some embodiments, R⁸ is tert-pentyl. In some embodiments, R⁷ is hexyl. In some embodiments, R⁸ is 4-methylpentyl. In some embodiments, R⁸ is 3-methylpentyl. In some embodiments, R⁸ is 2-methylpentyl. In some embodiments, R⁸ is hexan-2-yl. In some embodiments, R⁸ is 2,3-dimethylbutyl. In some embodiments, R⁸ is 4-methylpentan-2-yl. In some embodiments, R⁸ is 3-methylpentan-2-yl. In some embodiments, R⁸ is 2-ethylbutyl. In some embodiments, R⁸ is hexan-3-yl. In some embodiments, R⁸ is 3,3-dimethylbutyl. In some embodiments, R⁸ is 2,2-dimethylbutyl. In some embodiments, R⁸ is 2-methylpentan-2-yl. In some embodiments, R⁸ is C₂₋₆ alkenyl. In some embodiments, R⁸ is phenyl. In some embodiments, R⁸ is alkyl phenyl. In some embodiments, R⁸ is a C₁₋₆ alkyl phenyl group. In some embodiments, R⁸ is a C₂ alkyl phenyl group. In some embodiments, R⁸ is a naphthyl group. In some embodiments, R⁸ is a C₁₋₆ alkyl naphthyl group. In some embodiments, R⁸ is a C₂ alkyl naphthyl group. In some embodiments, R⁸ is a siloxy group. In some embodiments, R⁸ is a silyl group.

In some embodiments, R⁹ is hydrogen. In some embodiments, R⁹ is C₁₋₁₀ alkyl. In some embodiments, R⁹ is methyl. In some embodiments, R⁹ is ethyl. In some embodiments, R⁹ is propyl. In some embodiments, R⁹ is isopropyl. In some embodiments, R⁹ is butyl. In some embodiments, R⁹ is isobutyl. In some embodiments, R⁹ is sec-butyl. In some embodiments, R⁹ is tert-butyl. In some embodiments, R⁹ is pentyl. In some embodiments, R⁹ is isopentyl. In some embodiments, R⁹ is 2-methylbutyl. In some embodiments, R⁹ is pentan-2-yl. In some embodiments, R⁹ is 3-methylbutan-2-yl. In some embodiments, R⁹ is pentan-3-yl. In some embodiments, R⁹ is neopentyl. In some embodiments, R⁹ is tert-pentyl. In some embodiments, R⁷ is hexyl. In some embodiments, R⁹ is 4-methylpentyl. In some embodiments, R⁹ is 3-methylpentyl. In some embodiments, R⁹ is 2-methylpentyl. In some embodiments, R⁹ is hexan-2-yl. In some embodiments, R⁹ is 2,3-dimethylbutyl. In some embodiments, R⁹ is 4-methylpentan-2-yl. In some embodiments, R⁹ is 3-methylpentan-2-yl. In some embodiments, R⁹ is 2-ethylbutyl. In some embodiments, R⁹ is hexan-3-yl. In some embodiments, R⁹ is 3,3-dimethylbutyl. In some embodiments, R⁹ is 2,2-dimethylbutyl. In some embodiments, R⁹ is 2-methylpentan-2-yl. In some embodiments, R⁹ is C₂₋₆ alkenyl. In some embodiments, R⁹ is phenyl. In some embodiments, R⁹ is alkyl phenyl. In some embodiments, R⁹ is a C₁₋₆ alkyl phenyl group. In some embodiments, R⁹ is a C₂ alkyl phenyl group. In some embodiments, R⁹ is a naphthyl group. In some embodiments, R⁹ is a C₁₋₆ alkyl naphthyl group. In some embodiments, R⁹ is a C₂ alkyl naphthyl group. In some embodiments, R⁹ is a siloxy group. In some embodiments, R⁹ is a silyl group.

In some embodiments, two of R⁵, R⁶, R⁷, R⁸, and R⁹ are hydrogen. In some embodiments, three of R⁵, R⁶, R⁷, R⁸, and R⁹ are hydrogen. In some embodiments, four of R⁵, R⁶, R⁷, R⁸, and R⁹ are hydrogen.

In further embodiments where R¹ of formula (I) is

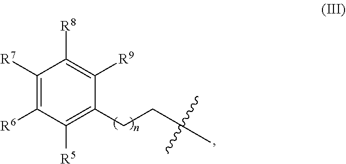

at least one of R⁵, R⁶, R⁷, R⁸, and R⁹ has the formula

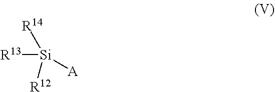

wherein A is at least one of R⁵, R⁶, R⁷, R⁸, and R⁹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group. In some embodiments, each of R¹², R¹³, and R¹⁴ have an alkyl and alkenyl group as described above. In some embodiments, R¹² is a phenyl group or an alkyl phenyl group. In some embodiments, R¹² is a naphthyl group or an alkyl naphthyl group. In some embodiments, R¹³ is a phenyl group or an alkyl phenyl group. In some embodiments, R¹³ is a naphthyl group or an alkyl naphthyl group. In some embodiments, R¹⁴ is a phenyl group or an alkyl phenyl group. In some embodiments, R¹⁴ is a naphthyl group or an alkyl naphthyl group. In some embodiments, R¹² is a silyl group or a siloxy group. In some embodiments, R¹³ is a silyl group or a siloxy group. In some embodiments, R¹⁴ is a silyl group or a siloxy group.

In some embodiments, R¹ of formula I is

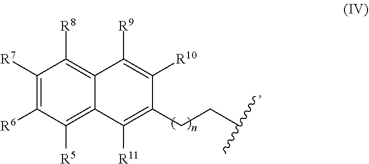

wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, a naphthyl group, or a siloxy group, with the proviso that at least one of R⁵, R⁶, R⁷, R⁸, R⁹, R¹⁰, and R¹¹ contains Si.

In some embodiments, R⁵ is hydrogen. In some embodiments, R⁵ is C₁₋₁₀ alkyl. In some embodiments, R⁵ is methyl. In some embodiments, R⁵ is ethyl. In some embodiments, R⁵ is propyl. In some embodiments, R⁵ is isopropyl. In some embodiments, R⁵ is butyl. In some embodiments, R⁵ is isobutyl. In some embodiments, R⁵ is sec-butyl. In some embodiments, R⁵ is tert-butyl. In some embodiments, R⁵ is pentyl. In some embodiments, R⁵ is isopentyl. In some embodiments, R⁵ is 2-methylbutyl. In some embodiments, R⁵ is pentan-2-yl. In some embodiments, R⁵ is 3-methylbutan-2-yl. In some embodiments, R⁵ is pentan-3-yl. In some embodiments, R⁵ is neopentyl. In some embodiments, R⁵ is tert-pentyl. In some embodiments, R⁵ is hexyl. In some embodiments, R⁵ is 4-methylpentyl. In some embodiments, R⁵ is 3-methylpentyl. In some embodiments, R⁵ is 2-methylpentyl. In some embodiments, R⁵ is hexan-2-yl. In some embodiments, R⁵ is 2,3-dimethylbutyl. In some embodiments, R⁵ is 4-methylpentan-2-yl. In some embodiments, R⁵ is 3-methylpentan-2-yl. In some embodiments, R⁵ is 2-ethylbutyl. In some embodiments, R⁵ is hexan-3-yl. In some embodiments, R⁵ is 3,3-dimethylbutyl. In some embodiments, R⁵ is 2,2-dimethylbutyl. In some embodiments, R⁵ is 2-methylpentan-2-yl. In some embodiments, R⁵ is C₂₋₆ alkenyl. In some embodiments, R⁵ is phenyl. In some embodiments, R⁵ is alkyl phenyl. In some embodiments, R⁵ is a C₁₋₆ alkyl phenyl group. In some embodiments, R⁵ is a C₂ alkyl phenyl group. In some embodiments, R⁵ is a naphthyl group. In some embodiments, R⁵ is a C₁₋₆ alkyl naphthyl group. In some embodiments, R⁵ is a C₂ alkyl naphthyl group. In some embodiments, R⁵ is a siloxy group. In some embodiments, R⁵ is a silyl group.

In some embodiments, R⁶ is hydrogen. In some embodiments, R⁶ is C₁₋₁₀ alkyl. In some embodiments, R⁶ is methyl. In some embodiments, R⁶ is ethyl. In some embodiments, R⁶ is propyl. In some embodiments, R⁶ is isopropyl. In some embodiments, R⁶ is butyl. In some embodiments, R⁶ is isobutyl. In some embodiments, R⁶ is sec-butyl. In some embodiments, R⁶ is tert-butyl. In some embodiments, R⁶ is pentyl. In some embodiments, R⁶ is isopentyl. In some embodiments, R⁶ is 2-methylbutyl. In some embodiments, R⁶ is pentan-2-yl. In some embodiments, R⁶ is 3-methylbutan-2-yl. In some embodiments, R⁶ is pentan-3-yl. In some embodiments, R⁶ is neopentyl. In some embodiments, R⁶ is tert-pentyl. In some embodiments, R⁶ is hexyl. In some embodiments, R⁶ is 4-methylpentyl. In some embodiments, R⁶ is 3-methylpentyl. In some embodiments, R⁶ is 2-methylpentyl. In some embodiments, R⁶ is hexan-2-yl. In some embodiments, R⁶ is 2,3-dimethylbutyl. In some embodiments, R⁶ is 4-methylpentan-2-yl. In some embodiments, R⁶ is 3-methylpentan-2-yl. In some embodiments, R⁶ is 2-ethylbutyl. In some embodiments, R⁶ is hexan-3-yl. In some embodiments, R⁶ is 3,3-dimethylbutyl. In some embodiments, R⁶ is 2,2-dimethylbutyl. In some embodiments, R⁶ is 2-methylpentan-2-yl. In some embodiments, R⁶ is C₂₋₆ alkenyl. In some embodiments, R⁶ is phenyl. In some embodiments, R⁶ is alkyl phenyl. In some embodiments, R⁶ is a C₁₋₆ alkyl phenyl group. In some embodiments, R⁶ is a C₂ alkyl phenyl group. In some embodiments, R⁶ is a naphthyl group. In some embodiments, R⁶ is a C₁₋₆ alkyl naphthyl group. In some embodiments, R⁶ is a C₂ alkyl naphthyl group. In some embodiments, R⁶ is a siloxy group. In some embodiments, R⁶ is a silyl group.

In some embodiments, R⁷ is hydrogen. In some embodiments, R⁷ is C₁₋₁₀ alkyl. In some embodiments, R⁷ is methyl. In some embodiments, R⁷ is ethyl. In some embodiments, R⁷ is propyl. In some embodiments, R⁷ is isopropyl. In some embodiments, R⁷ is butyl. In some embodiments, R⁷ is isobutyl. In some embodiments, R⁷ is sec-butyl. In some embodiments, R⁷ is tert-butyl. In some embodiments, R⁷ is pentyl. In some embodiments, R⁷ is isopentyl. In some embodiments, R⁷ is 2-methylbutyl. In some embodiments, R⁷ is pentan-2-yl. In some embodiments, R⁷ is 3-methylbutan-2-yl. In some embodiments, R⁷ is pentan-3-yl. In some embodiments, R⁷ is neopentyl. In some embodiments, R⁷ is tert-pentyl. In some embodiments, R⁷ is hexyl. In some embodiments, R⁷ is 4-methylpentyl. In some embodiments, R⁷ is 3-methylpentyl. In some embodiments, R⁷ is 2-methylpentyl. In some embodiments, R⁷ is hexan-2-yl. In some embodiments, R⁷ is 2,3-dimethylbutyl. In some embodiments, R⁷ is 4-methylpentan-2-yl. In some embodiments, R⁷ is 3-methylpentan-2-yl. In some embodiments, R⁷ is 2-ethylbutyl. In some embodiments, R⁷ is hexan-3-yl. In some embodiments, R⁷ is 3,3-dimethylbutyl. In some embodiments, R⁷ is 2,2-dimethylbutyl. In some embodiments, R⁷ is 2-methylpentan-2-yl. In some embodiments, R⁷ is C₂₋₆ alkenyl. In some embodiments, R⁷ is phenyl. In some embodiments, R⁷ is alkyl phenyl. In some embodiments, R⁷ is a C₁₋₆ alkyl phenyl group. In some embodiments, R⁷ is a C₂ alkyl phenyl group. In some embodiments, R⁷ is a naphthyl group. In some embodiments, R⁷ is a C₁₋₆ alkyl naphthyl group. In some embodiments, R⁷ is a C₂ alkyl naphthyl group. In some embodiments, R⁷ is a siloxy group. In some embodiments, R⁷ is a silyl group.

In some embodiments, R⁸ is hydrogen. In some embodiments, R⁸ is C₁₋₁₀ alkyl. In some embodiments, R⁸ is methyl. In some embodiments, R⁸ is ethyl. In some embodiments, R⁸ is propyl. In some embodiments, R⁸ is isopropyl. In some embodiments, R⁸ is butyl. In some embodiments, R⁸ is isobutyl. In some embodiments, R⁸ is sec-butyl. In some embodiments, R⁸ is tert-butyl. In some embodiments, R⁸ is pentyl. In some embodiments, R⁸ is isopentyl. In some embodiments, R⁸ is 2-methylbutyl. In some embodiments, R⁸ is pentan-2-yl. In some embodiments, R⁸ is 3-methylbutan-2-yl. In some embodiments, R⁸ is pentan-3-yl. In some embodiments, R⁸ is neopentyl. In some embodiments, R⁸ is tert-pentyl. In some embodiments, R⁷ is hexyl. In some embodiments, R⁸ is 4-methylpentyl. In some embodiments, R⁸ is 3-methylpentyl. In some embodiments, R⁸ is 2-methylpentyl. In some embodiments, R⁸ is hexan-2-yl. In some embodiments, R⁸ is 2,3-dimethylbutyl. In some embodiments, R⁸ is 4-methylpentan-2-yl. In some embodiments, R⁸ is 3-methylpentan-2-yl. In some embodiments, R⁸ is 2-ethylbutyl. In some embodiments, R⁸ is hexan-3-yl. In some embodiments, R⁸ is 3,3-dimethylbutyl. In some embodiments, R⁸ is 2,2-dimethylbutyl. In some embodiments, R⁸ is 2-methylpentan-2-yl. In some embodiments, R⁸ is C₂₋₆ alkenyl. In some embodiments, R⁸ is phenyl. In some embodiments, R⁸ is alkyl phenyl. In some embodiments, R⁸ is a C₁₋₆ alkyl phenyl group. In some embodiments, R⁸ is a C₂ alkyl phenyl group. In some embodiments, R⁸ is a naphthyl group. In some embodiments, R⁸ is a C₁₋₆ alkyl naphthyl group. In some embodiments, R⁸ is a C₂ alkyl naphthyl group. In some embodiments, R⁸ is a siloxy group. In some embodiments, R⁸ is a silyl group.

In some embodiments, R⁹ is hydrogen. In some embodiments, R⁹ is C₁₋₁₀ alkyl. In some embodiments, R⁹ is methyl. In some embodiments, R⁹ is ethyl. In some embodiments, R⁹ is propyl. In some embodiments, R⁹ is isopropyl. In some embodiments, R⁹ is butyl. In some embodiments, R⁹ is isobutyl. In some embodiments, R⁹ is sec-butyl. In some embodiments, R⁹ is tert-butyl. In some embodiments, R⁹ is pentyl. In some embodiments, R⁹ is isopentyl. In some embodiments, R⁹ is 2-methylbutyl. In some embodiments, R⁹ is pentan-2-yl. In some embodiments, R⁹ is 3-methylbutan-2-yl. In some embodiments, R⁹ is pentan-3-yl. In some embodiments, R⁹ is neopentyl. In some embodiments, R⁹ is tert-pentyl. In some embodiments, R⁷ is hexyl. In some embodiments, R⁹ is 4-methylpentyl. In some embodiments, R⁹ is 3-methylpentyl. In some embodiments, R⁹ is 2-methylpentyl. In some embodiments, R⁹ is hexan-2-yl. In some embodiments, R⁹ is 2,3-dimethylbutyl. In some embodiments, R⁹ is 4-methylpentan-2-yl. In some embodiments, R⁹ is 3-methylpentan-2-yl. In some embodiments, R⁹ is 2-ethylbutyl. In some embodiments, R⁹ is hexan-3-yl. In some embodiments, R⁹ is 3,3-dimethylbutyl. In some embodiments, R⁹ is 2,2-dimethylbutyl. In some embodiments, R⁹ is 2-methylpentan-2-yl. In some embodiments, R⁹ is C₂₋₆ alkenyl. In some embodiments, R⁹ is a phenyl group. In some embodiments, R⁹ is a naphthyl group. In some embodiments, R⁹ is a siloxy group.

In some embodiments, R¹⁰ is hydrogen. In some embodiments, R¹⁰ is C₁₋₁₀ alkyl. In some embodiments, R¹⁰ is methyl. In some embodiments, R¹⁰ is ethyl. In some embodiments, R¹⁰ is propyl. In some embodiments, R¹⁰ is isopropyl. In some embodiments, R¹⁰ is butyl. In some embodiments, R¹⁰ is isobutyl. In some embodiments, R¹⁰ is sec-butyl. In some embodiments, R¹⁰ is tert-butyl. In some embodiments, R¹⁰ is pentyl. In some embodiments, R¹⁰ is isopentyl. In some embodiments, R¹⁰ is 2-methylbutyl. In some embodiments, R¹⁰ is pentan-2-yl. In some embodiments, R¹⁰ is 3-methylbutan-2-yl. In some embodiments, R¹⁰ is pentan-3-yl. In some embodiments, R¹⁰ is neopentyl. In some embodiments, R¹⁰ is tert-pentyl. In some embodiments, R¹⁰ is hexyl. In some embodiments, R¹⁰ is 4-methylpentyl. In some embodiments, R¹⁰ is 3-methylpentyl. In some embodiments, R¹⁰ is 2-methylpentyl. In some embodiments, R¹⁰ is hexan-2-yl. In some embodiments, R¹⁰ is 2,3-dimethylbutyl. In some embodiments, R¹⁰ is 4-methylpentan-2-yl. In some embodiments, R¹⁰ is 3-methylpentan-2-yl. In some embodiments, R¹⁰ is 2-ethylbutyl. In some embodiments, R¹⁰ is hexan-3-yl. In some embodiments, R¹⁰ is 3,3-dimethylbutyl. In some embodiments, R¹⁰ is 2,2-dimethylbutyl. In some embodiments, R¹⁰ is 2-methylpentan-2-yl. In some embodiments, R¹⁰ is C₂₋₆ alkenyl. In some embodiments, R¹⁰ is phenyl. In some embodiments, R¹⁰ is alkyl phenyl. In some embodiments, R¹⁰ is a C₁₋₆ alkyl phenyl group. In some embodiments, R¹⁰ is a C₂ alkyl phenyl group. In some embodiments, R¹⁰ is a naphthyl group. In some embodiments, R¹⁰ is a C₁₋₆ alkyl naphthyl group. In some embodiments, R¹⁰ is a C₂ alkyl naphthyl group. In some embodiments, R¹⁰ is a siloxy group. In some embodiments, R¹⁰ is a silyl group.

In some embodiments, R¹¹ is hydrogen. In some embodiments, R¹¹ is C₁₋₁₀ alkyl. In some embodiments, R¹¹ is methyl. In some embodiments, R¹¹ is ethyl. In some embodiments, R¹¹ is propyl. In some embodiments, R¹¹ is isopropyl. In some embodiments, R¹¹ is butyl. In some embodiments, R¹¹ is isobutyl. In some embodiments, R¹¹ is sec-butyl. In some embodiments, R¹¹ is tert-butyl. In some embodiments, R¹¹ is pentyl. In some embodiments, R¹¹ is isopentyl. In some embodiments, R¹¹ is 2-methylbutyl. In some embodiments, R¹¹ is pentan-2-yl. In some embodiments, R¹¹ is 3-methylbutan-2-yl. In some embodiments, R¹¹ is pentan-3-yl. In some embodiments, R¹¹ is neopentyl. In some embodiments, R¹¹ is tert-pentyl. In some embodiments, R⁷ is hexyl. In some embodiments, R¹¹ is 4-methylpentyl. In some embodiments, R¹¹ is 3-methylpentyl. In some embodiments, R¹¹ is 2-methylpentyl. In some embodiments, R¹¹ is hexan-2-yl. In some embodiments, R¹¹ is 2,3-dimethylbutyl. In some embodiments, R¹¹ is 4-methylpentan-2-yl. In some embodiments, R¹¹ is 3-methylpentan-2-yl. In some embodiments, R¹¹ is 2-ethylbutyl. In some embodiments, R¹¹ is hexan-3-yl. In some embodiments, R¹¹ is 3,3-dimethylbutyl. In some embodiments, R¹¹ is 2,2-dimethylbutyl. In some embodiments, R¹¹ is 2-methylpentan-2-yl. In some embodiments, R¹¹ is C₂₋₆ alkenyl. In some embodiments, R¹¹ is phenyl. In some embodiments, R¹¹ is alkyl phenyl. In some embodiments, R¹¹ is a C₁₋₆ alkyl phenyl group. In some embodiments, R¹¹ is a C₂ alkyl phenyl group. In some embodiments, R¹¹ is a naphthyl group. In some embodiments, R¹¹ is a C₁₋₆ alkyl naphthyl group. In some embodiments, R¹¹ is a C₂ alkyl naphthyl group. In some embodiments, R¹¹ is a siloxy group. In some embodiments, R¹¹ is a silyl group.

In some embodiments, two of R⁵, R⁶, R⁷, R⁸, R⁹, R¹⁰, and R¹¹ are hydrogen. In some embodiments, three of R⁵, R⁶, R⁷, R⁸, R⁹, R¹⁰, and R¹¹ are hydrogen. In some embodiments, four of R⁵, R⁶, R⁷, R⁸, R⁹, R¹⁰, and R¹¹ are hydrogen. In some embodiments, five of R⁵, R⁶, R⁷, R⁸, R⁹, R¹⁰, and R¹¹ are hydrogen. In some embodiments, six of R⁵, R⁶, R⁷, R⁸, R⁹, R¹⁰, and R¹¹ are hydrogen.

In further embodiments where R¹ of formula (I) is

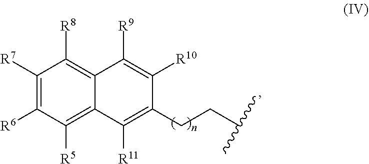

at least one of R⁵, R⁶, R⁷, R⁸, R⁹, R¹⁰, and R¹¹ has the formula:

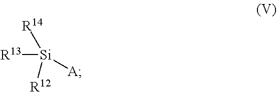

wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group. In some embodiments, each of R¹², R¹³, and R¹⁴ have an alkyl and alkenyl group as described above. In some embodiments, R¹² is a phenyl group or an alkyl phenyl group. In some embodiments, R¹² is a naphthyl group or an alkyl naphthyl group. In some embodiments, R¹³ is a phenyl group or an alkyl phenyl group. In some embodiments, R¹³ is a naphthyl group or an alkyl naphthyl group. In some embodiments, R¹⁴ is a phenyl group or an alkyl phenyl group. In some embodiments, R¹⁴ is a naphthyl group or an alkyl naphthyl group. In some embodiments, R¹² is a silyl group or a siloxy group. In some embodiments, R¹³ is a silyl group or a siloxy group. In some embodiments, R¹⁴ is a silyl group or a siloxy group.

In some embodiments, each R group specified above may also comprise silylcycloalkyl groups, such as silylcyclo C₁₋₁₀ alkyl, including cyclobutyl.

In some aspects, the novel silyl-containing AHL is (S)—N-(2-oxotetrahydrofuran-3-yl)-5-(triethylsilyl)pentanamide; (S)-5-(dimethyl(octyl)silyl)-N-(2-oxotetrahydrofuran-3-yl)pentanamide; (S)-2-(4-(butyldimethylsilyl)phenyl)-N-(2-oxotetrahydrofuran-3-yl)acetamide; (S)-3-oxo-N-(2-oxotetrahydrofuran-3-yl)-7-(triethylsilyl)heptanamide; (S)-2-(4-(dimethyl(phenyl)silyl)phenyl)-N-(2-oxotetrahydrofuran-3-yl)acetamide; (S)—N-(2-oxocyclopentyl)-2-(4-(triethylsilyl)phenyl)acetamide; (S)-5-(dimethyl(phenyl)silyl)-N-(2-oxotetrahydrofuran-3-yl)pentanamide; (S)-5-(butyldimethylsilyl)-N-(2-oxotetrahydrofuran-3-yl)pentanamide; (S)—N-(2-oxotetrahydrofuran-3-yl)-5-(tributylsilyl)pentanamide; (S)—N-(2-oxotetrahydrofuran-3-yl)-5-(1,1,3,3,3-pentamethyldisiloxaneyl)pentanamide; (S)-7-(dimethyl(phenyl)silyl)-N-(2-oxotetrahydrofuran-3-yl)heptanamide; (S)-7-(butyldimethylsilyl)-N-(2-oxotetrahydrofuran-3-yl)heptanamide; (S)—N-(2-oxotetrahydrofuran-3-yl)-7-(triethylsilyl)heptanamide; (S)-7-(dimethyl(phenyl)silyl)-3-oxo-N-(2-oxotetrahydrofuran-3-yl)heptanamide; (S)-7-(butyldimethylsilyl)-3-oxo-N-(2-oxotetrahydrofuran-3-yl)heptanamide; (S)-2-(4-(dimethyl(octyl)silyl)phenyl)-N-(2-oxotetrahydrofuran-3-yl)acetamide; or (S)-7-(dimethyl(octyl)silyl)-N-(2-oxotetrahydrofuran-3-yl)heptanamide.

The compounds provided herein may also be the salts, solvates, and isomers of any on the structures disclosed above. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates, and salts with amino acids such as glutamic acid. These salts may be prepared by methods known to those skilled in art. When compounds of the present disclosure contain relatively basic functionalities, acid addition salts may be obtained by contacting the neutral form of such compounds with a sufficient amount of the desired acid, either neat or in a suitable inert solvent. Examples of acceptable acid addition salts include those derived from inorganic acids such as hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids such as acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

Also included are salts of amino acids such as arginate and the like, and salts of organic acids such as glucuronic or galactunoric acids and the like (see, for example, Berge et al., Journal of Pharmaceutical Science 66, (1977):1). Certain specific compounds of the present disclosure contain basic acidic functionalities that allow the compounds to be converted into base addition salts. Additional information on suitable pharmaceutically acceptable salts can be found in Remington's Pharmaceutical Sciences, 17th ed., Mack Publishing Company, Easton, Pa., 1985, which is incorporated herein by reference.

The neutral forms of the compounds are preferably regenerated by contacting the salt with a base or acid and isolating the parent compound in the conventional manner. he parent form of the compound differs from the various salt forms in certain physical properties, such as solubility in polar solvents.

Certain compounds of the present disclosure may exist in unsolvated forms as well as solvated forms, including hydrated forms. In general, the solvated forms are equivalent to unsolvated forms and are encompassed within the scope of the present disclosure. Certain compounds of the present invention may exist in multiple crystalline or amorphous forms. In general, all physical forms are equivalent for the uses contemplated by the present disclosure and are intended to be within the scope of the present disclosure.

Certain compounds of the present disclosure possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present disclosure may also contain unnatural proportions of atomic isotopes at one or more of the atoms that constitute such compounds. For example, the compounds of the present disclosure may be radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of the present disclosure, whether radioactive or not, are encompassed within the scope of the present invention.

The present invention further includes compounds which are in a prodrug form. Prodrugs of the compounds described herein are those compounds that readily undergo chemical changes under physiological conditions to provide any of the compounds disclosed above. Additionally, prodrugs can be converted to the compounds of the present disclosure by chemical or biochemical methods in an ex vivo environment. For example, prodrugs can be slowly converted to the compounds of the present invention when placed in a transdermal patch reservoir with a suitable enzyme or chemical reagent.

### Pharmaceutical Compositions

In another aspect, the present disclosure provides pharmaceutical compositions including one or more pharmaceutically acceptable carriers, diluents, excipients, or buffers and one or more of the compounds provided herein. In some embodiments, the pharmaceutically acceptable carrier, diluent, excipient, or buffer is suitable for use in a subject, for example, a human. Pharmaceutically acceptable excipients include, but are not limited to, liquids such as water, saline, glycerol, sugars and ethanol. The preparation of pharmaceutically acceptable carriers and excipients is described in, e.g., Remington: The Science and Practice of Pharmacy, 22nd edition, Loyd V. Allen et al, editors, Pharmaceutical Press (2012).

In some embodiments, the composition also includes an additional active compound or other chemotherapeutic agent. In some embodiments, the pharmaceutical composition further includes one or more stabilizing compounds, which may be administered in any sterile, biocompatible pharmaceutical carrier, including, but not limited to, saline, buffered saline, dextrose, and water. In some embodiments, the pharmaceutical compositions also contain a pharmaceutically acceptable salt. Pharmaceutically acceptable salts can include, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in the provided pharmaceutical compositions. The pharmaceutical compositions may be subjected to conventional pharmaceutical operations such as sterilization.

### Formulations

The compositions provided herein can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by a subject, e.g., a human patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35, (1995):1187-1193; Tjwa, Ann. Allergy Asthma Immunol. 75, (1995):107).

For preparing pharmaceutical formulations including the compounds of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% by weight of the compounds of the present disclosure.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention may also be used orally using, for example, push-fit capsules made of gelatin, as well as soft, sealed capsules made of gelatin and a coating such as glycerol or sorbitol. Push-fit capsules may contain the compounds of the present invention mixed with a filler or binders such as lactose or starches, lubricants such as talc or magnesium stearate, and, optionally, stabilizers. In soft capsules, the compounds of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compounds of the present disclosure are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations may be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use may be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension may also contain one or more preservatives such as ethyl or n-propyl p-hydroxybenzoate, one or more coloring agents, one or more flavoring agents and one or more sweetening agents, such as sucrose, aspartame or saccharin. Formulations may be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions may be formulated by suspending the compounds of the present invention in a vegetable oil, such as arachis oil, olive oil, sesame oil, or coconut oil; or in a mineral oil such as liquid paraffin; or a mixture of these. The oil suspensions may contain a thickening agent, such as beeswax, hard paraffin, or cetyl alcohol. Sweetening agents, such as glycerol, sorbitol or sucrose, may be added to provide a palatable oral preparation. These formulations may be preserved by the addition of an antioxidant such as ascorbic acid. As an example of an injectable oil vehicle, see Minto, J. Pharmacol. Exp. Ther. 281, (1997):93. The pharmaceutical formulations of the disclosure may also be in the form of oil-in-water emulsions. The oily phase may be a vegetable oil or a mineral oil, described above, or a mixture of these. Suitable emulsifying agents include naturally occurring gums such as gum acacia and gum tragacanth, naturally occurring phosphatides such as soybean lecithin, esters or partial esters derived from fatty acids and hexitol anhydrides such as sorbitan mono-oleate, and condensation products of these partial esters with ethylene oxide such as polyoxyethylene sorbitan mono-oleate. The emulsion may also contain sweetening agents and flavoring agents, as in the formulation of syrups and elixirs. Such formulations may also contain a demulcent, a preservative, or a coloring agent.

The compositions of the present invention may also be delivered as microspheres for slow release in the body. For example, microspheres may be formulated for administration via intradermal injection of drug-containing microspheres, which slowly release subcutaneously (see Rao, J. Biomater Sci. Polym. Ed. 7, (1995):623); as biodegradable and injectable gel formulations (see, e.g., Gao Pharm. Res. 12, (1995):857); or as microspheres for oral administration (see, e.g., Eyles, J. Pharm. Pharmacol. 49, (1997):669). Both transdermal and intradermal routes may afford constant delivery for weeks or months.

In other embodiments, the compositions of the present disclosure are formulated for parenteral administration, such as intravenous (IV) administration or administration into a body cavity or lumen of an organ. Such formulations for administration will commonly comprise a solution of the compositions of the present disclosure dissolved in a pharmaceutically acceptable carrier. Among the acceptable vehicles and solvents that may be employed are water and Ringer's solution, an isotonic sodium chloride. In addition, sterile fixed oils may conventionally be employed as a solvent or suspending medium. For this purpose, any bland fixed oil may can be employed including synthetic mono- or diglycerides. In addition, fatty acids such as oleic acid may likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH-adjusting and buffering agents, toxicity adjusting agents, e.g., sodium acetate, sodium chloride, potassium chloride, calcium chloride, sodium lactate and the like. The concentration of the compositions of the present disclosure in these formulations may vary widely, and will be selected primarily based on fluid volumes, viscosities, body weight, and the like, in accordance with the particular mode of administration selected and the patient's needs. For IV administration, the formulation may be a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a nontoxic parenterally acceptable diluent or solvent, such as a solution of 1,3-butanediol.

In another embodiment, formulations of the compositions of the present disclosure are delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, delivery may be focused into the target cells in vivo. (See, e.g., Al-Muhammed, J. Microencapsul. 13, (1996):293; Chonn, Curr. Opin. Biotechnol. 6, (1995):698; Ostro, Am. J. Hosp. Pharm. 46, (1989):1576-1587).

Lipid-based formulations include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present disclosure include any natural or synthetic lipids including, but not limited to, sesame seed oil, olive oil, castor oil, peanut oil, fatty acid esters, glycerol esters, LABRAFIL®, LABRASOL®, CREMOPHOR®, SOLUTOL®, TWEEN®, CAPRYOL®, CAPMUL®, CAPTEX®, and PECEOL®.

### Methods of Preparing Compounds

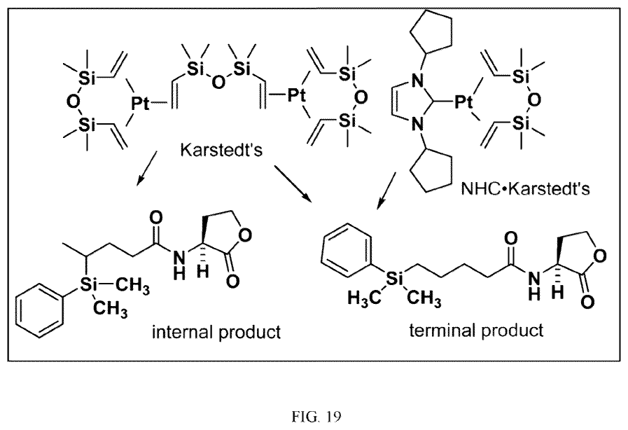

The present disclosure provides methods for preparing the compounds disclosed herein. Generally, the compounds may be synthesized using two complementary amidation conditions: Schotten Baumann that allows for the conversion of carboxylic acid to amide in a 1:1 DCM:Water mix and EDT/HOBt. When X is an oxygen in Formula II, then several additional protection and deprotection steps may be needed in the synthesis. The compound may then be hydrosilylated according to the scheme shown in FIG. 19, and as described in Dierick, S.; Markó, I. E. NHC Platinum(0) Complexes: Unique Catalysts for the Hydrosilylation of Alkenes and Alkynes. N-Heterocyclic Carbenes Eff Tools Organomet. Synth. 2014, 9783527334 (0), 111-150. For some compounds, instead of using a hydrosilylation reaction, an aryl iodide compound undergoes aryl silylation to install a silyl group as described in Rhodium(I)-Catalyzed Silylation of Aryl Halides with Triethoxysilane: Practical Synthetic Route to Aryltriethoxysilanes Org. Lett., Vol. 4, No. 11, 2002. The conditions may vary between aryl and alkyl silane hydrosilylation and the hydrosilylation and silylation catalysts may be selected based on the yield and isomeric purity of the products. In some aspects, a metal hydrosilylation catalyst may be used. In some aspects, a platinum hydrosilylation catalyst may be used. For example, the platinum hydrosilylation catalyst may be a platinum oxide catalyst or a Karstedt catalyst.

### EXEMPLARY EMBODIMENTS

The following embodiments are contemplated. All combinations of features and embodiments are contemplated.

Embodiment 1. A compound having the formula:

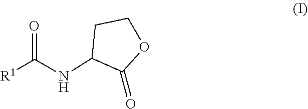

wherein R¹ has the formula:

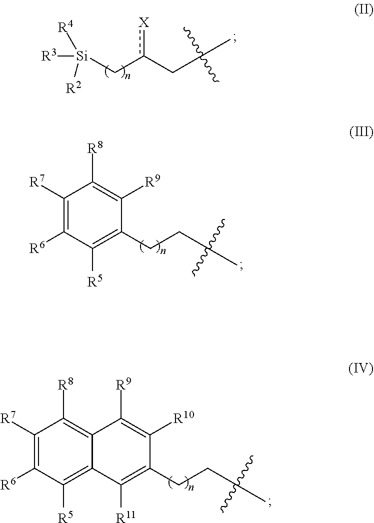

wherein R²; R³, and R⁴ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group;
wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group, with the proviso that at least one of R⁵, R⁶, R⁷, R⁸, R⁹, R¹⁰, and R¹¹ comprises Si;
wherein X is hydrogen, or oxygen,
wherein the subscript n is an integer from 0 to 9;
or a pharmaceutically acceptable salt, solvate, or isomer thereof.

Embodiment 2. The compound of Embodiment 1, wherein R¹ is Formula (II).

Embodiment 3. The compound of any of the preceding Embodiments, wherein R¹ is Formula (II) and wherein R², R³, and R⁴ are independently selected from a C₁₋₁₀ alkyl group.

Embodiment 4. The compound of any of the preceding Embodiments, wherein R¹ is Formula (II) and wherein R², R³, and R⁴ are independently selected from a C₁₋₅ alkyl group.

Embodiment 5. The compound of any of the preceding Embodiments, wherein R¹ is Formula (II) and wherein at least two of R², R³, and R⁴ are identical.

Embodiment 6. The compound of Embodiment 1, wherein R¹ is Formula (II) and wherein R², R³, and R⁴ are identical and are selected from a C₁₋₁₀ alkyl group.

Embodiment 7. The compound of any of Embodiments 1-2, wherein at least one of R², R³, and R⁴ is a phenyl group or an alkyl phenyl group.

Embodiment 8. The compound of Embodiment 1, wherein R¹ is Formula (III).

Embodiment 9. The compound of Embodiment 1 or 8, wherein two of R⁵, R⁶, R⁷, R⁸, and R⁹ are hydrogen.

Embodiment 10. The compound of Embodiment 1 or 8, wherein three of R⁵, R⁶, R⁷, R⁸, and R⁹ are hydrogen.

Embodiment 11. The compound of Embodiment 1 or 8, wherein four of R⁵, R⁶, R⁷, R⁸, and R⁹ are hydrogen.

Embodiment 12. The compound of Embodiment 1 or 8-11, wherein at least one of R⁵, R⁶, R⁷, R⁸, and R⁹ has the formula:

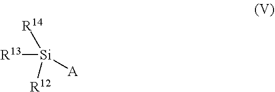

wherein A is at least one of R⁵, R⁶, R⁷, R⁸, and R⁹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

Embodiment 13. The compound of Embodiment 12, wherein at least one of R¹, R¹³ and R¹⁴ is a phenyl group or an alkyl phenyl group.

Embodiment 14. The compound of Embodiment 12 or 13, wherein at least one of R¹², R¹³, and R¹⁴ is an alkyl group.

Embodiment 15. The compound of Embodiment 1, wherein R¹ is Formula (IV).

Embodiment 16. The compound of Embodiment 1 or 15, wherein two of R⁵, R⁶, R⁷, R⁸, R⁹, R¹⁰ and R¹¹ are hydrogen.

Embodiment 17. The compound of Embodiment 1 or 15, wherein three of R⁵, R⁶, R⁷, R⁸, R⁹, R¹⁰ and R¹¹ are hydrogen.

Embodiment 18. The compound of Embodiment 1 or 15, wherein four of R⁵, R⁶, R⁷, R⁸, R⁹, R¹⁰ and R¹¹ are hydrogen.

Embodiment 19. The compound of Embodiment 1 or 15, wherein five of R⁵, R⁶, R⁷, R⁸, R⁹, R¹⁰ and R¹¹ are hydrogen.

Embodiment 20. The compound of Embodiment 1 or 15, wherein six of R⁵, R⁶, R⁷, R⁸, R⁹, R¹⁰ and R¹¹ are hydrogen.

Embodiment 21. The compound of Embodiment 1 or 15-20, wherein at least one of R⁵, R⁶, R⁷, R⁸, R⁹, R¹⁰ and R¹¹ has the formula:

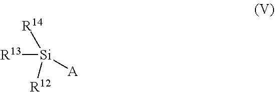

wherein A is at least one of R⁵, R⁶, R⁷, R⁸, R⁹, R¹⁰ and R¹¹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

Embodiment 22. The compound of Embodiment 21, wherein at least one of R¹², R¹³, and R¹⁴ is a phenyl group.

Embodiment 23. The compound of Embodiment 21 or 22, wherein at least one of R¹², R¹³, and R¹⁴ is an alkyl group.

Embodiment 24. The compound of any of the preceding Embodiments, wherein X is O.

Embodiment 25. The compound of any of Embodiments 1-23, wherein X is H.

Embodiment 26. The compound of Embodiment 1, wherein the compound is (S)—N-(2-oxotetrahydrofuran-3-yl)-5-(triethylsilyl)pentanamide.

Embodiment 27. The compound of Embodiment 1, wherein the compound is (S)-5-(dimethyl(octyl)silyl)-N-(2-oxotetrahydrofuran-3-yl)pentanamide.

Embodiment 28. The compound of Embodiment 1, wherein the compound is (S)-2-(4-(butyldimethylsilyl)phenyl)-N-(2-oxotetrahydrofuran-3-yl)acetamide.

Embodiment 29. The compound of Embodiment 1, wherein the compound is (S)-3-oxo-N-(2-oxotetrahydrofuran-3-yl)-7-(triethylsilyl)heptanamide.

Embodiment 30. The compound of Embodiment 1, wherein the compound is (S)-2-(4-(dimethyl(phenyl)silyl)phenyl)-N-(2-oxotetrahydrofuran-3-yl)acetamide.

Embodiment 31. The compound of Embodiment 1, wherein the compound is (S)—N-(2-oxocyclopentyl)-2-(4-(triethylsilyl)phenyl)acetamide.

Embodiment 32. The compound of Embodiment 1, wherein the compound is (S)-5-(dimethyl(phenyl)silyl)-N-(2-oxotetrahydrofuran-3-yl)pentanamide.

Embodiment 33. The compound of Embodiment 1, wherein the compound is (S)-5-(butyldimethylsilyl)-N-(2-oxotetrahydrofuran-3-yl)pentanamide.

Embodiment 34. The compound of Embodiment 1, wherein the compound is (S)—N-(2-oxotetrahydrofuran-3-yl)-5-(tributylsilyl)pentanamide.

Embodiment 35. The compound of Embodiment 1, wherein the compound is(S)—N-(2-oxotetrahydrofuran-3-yl)-5-(1,1,3,3,3-pentamethyldisiloxaneyl)pentanamide.

Embodiment 36. The compound of Embodiment 1, wherein the compound is (S)-7-(dimethyl(phenyl)silyl)-N-(2-oxotetrahydrofuran-3-yl)heptanamide.

Embodiment 37. The compound of Embodiment 1, wherein the compound is (S)-7-(butyldimethylsilyl)-N-(2-oxotetrahydrofuran-3-yl)heptanamide.

Embodiment 38. The compound of Embodiment 1, wherein the compound is (S)—N-(2-oxotetrahydrofuran-3-yl)-7-(triethylsilyl)heptanamide.

Embodiment 39. The compound of Embodiment 1, wherein the compound is (S)-7-(dimethyl(phenyl)silyl)-3-oxo-N-(2-oxotetrahydrofuran-3-yl)heptanamide.

Embodiment 40. The compound of Embodiment 1, wherein the compound is (S)-7-(butyldimethylsilyl)-3-oxo-N-(2-oxotetrahydrofuran-3-yl)heptanamide.

Embodiment 41. The compound of claim 1, wherein the compound is (S)-2-(4-(dimethyl(octyl)silyl)phenyl)-N-(2-oxotetrahydrofuran-3-yl)acetamide.

Embodiment 42. The compound of claim 1, wherein the compound is (S)-7-(dimethyl(octyl)silyl)-N-(2-oxotetrahydrofuran-3-yl)heptanamide.

Embodiment 43. Use of the compound of Embodiments 1-42 for quorum sensing.

### EXAMPLES

The present disclosure will be better understood in view of the following non-limiting examples. The following examples are intended for illustrative purposes only and do not limit in any way the scope of the present inventio

### Example 1

15 compounds were assayed for LasR agonism using a beta-glo reporter assay in E. coli. The compounds are shown below, along with EC₅₀, 95% Cl, and Maximum Activation (%), if applicable. EC₅₀ is the half maximal effective concentration which to the concentration of a drug, antibody or toxicant which induces a response halfway between the baseline and maximum after a specified exposure time.

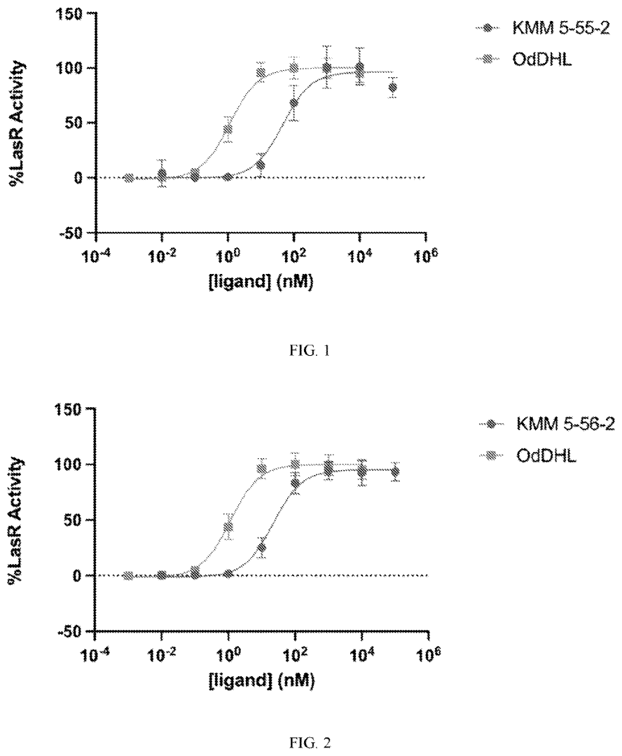

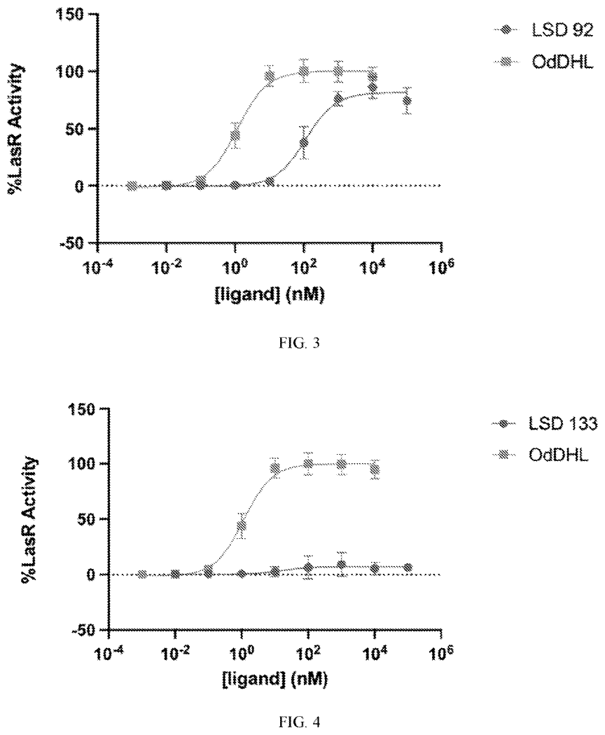

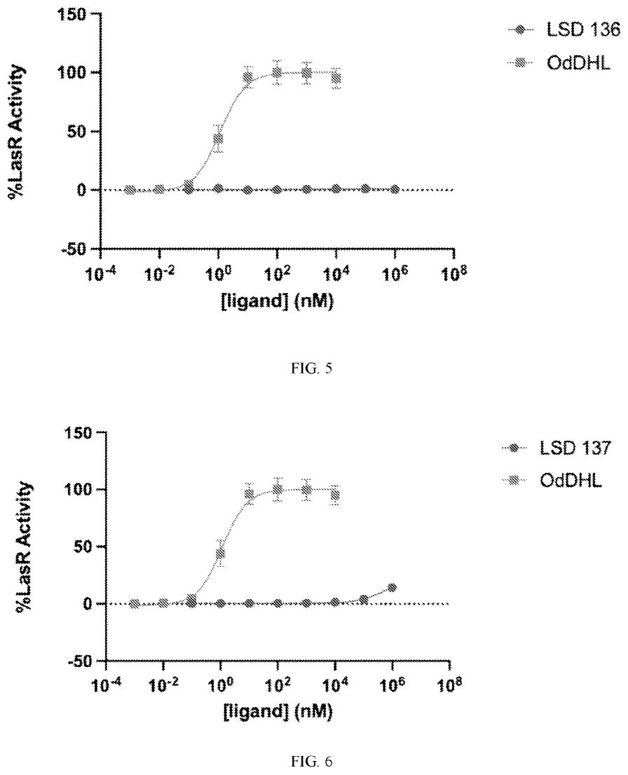

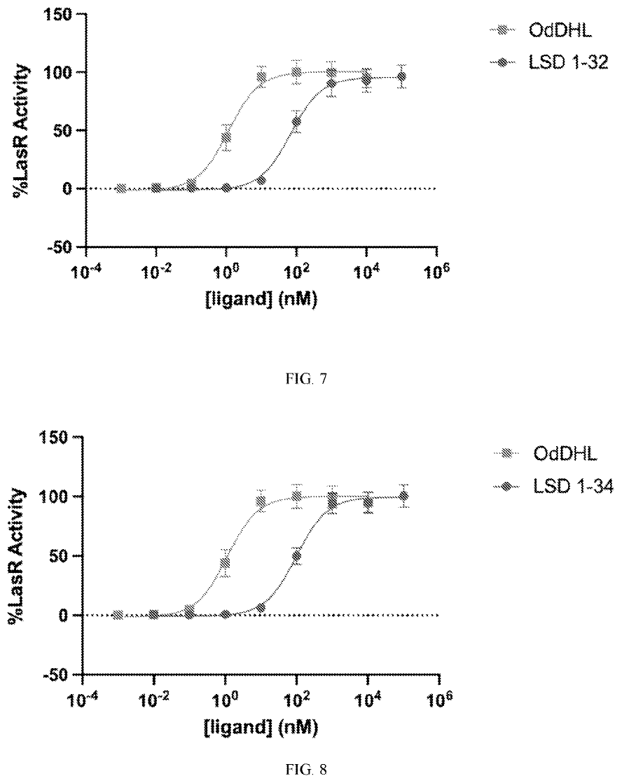

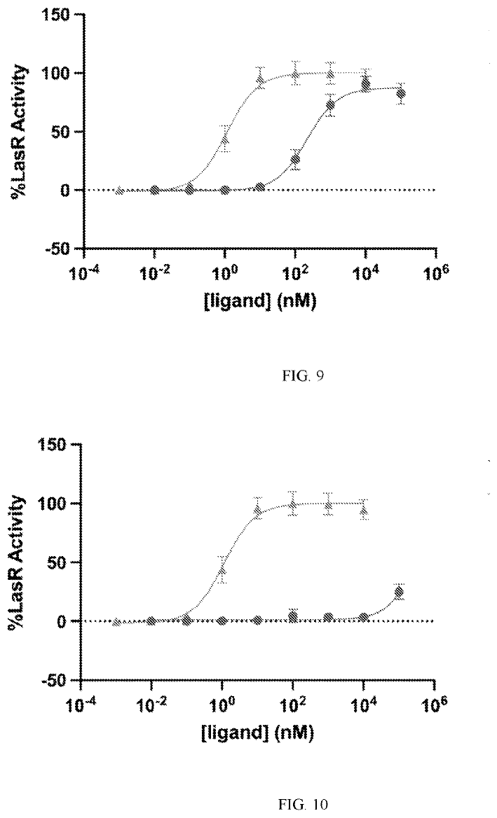

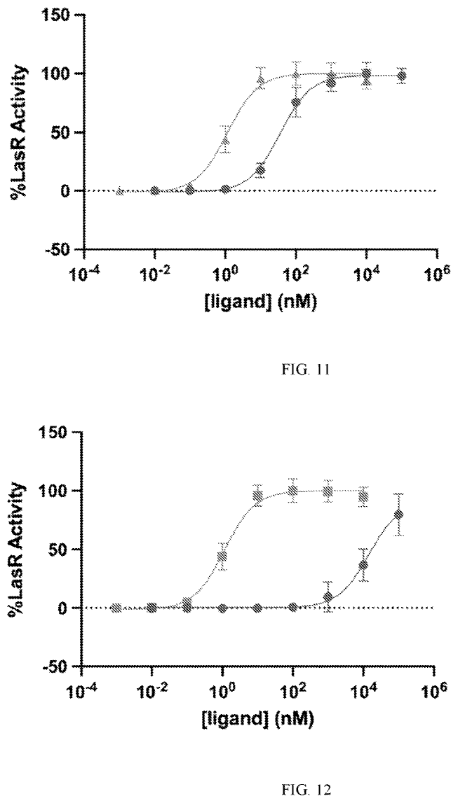

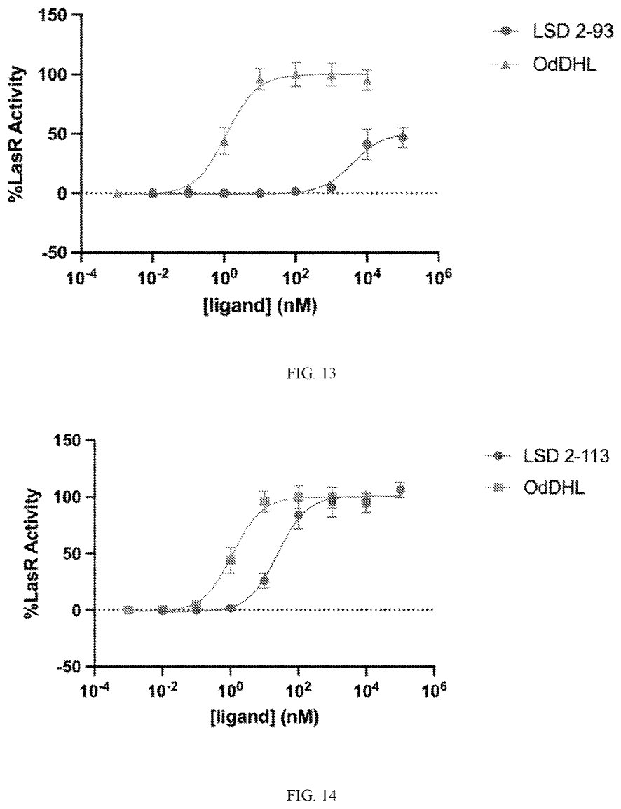

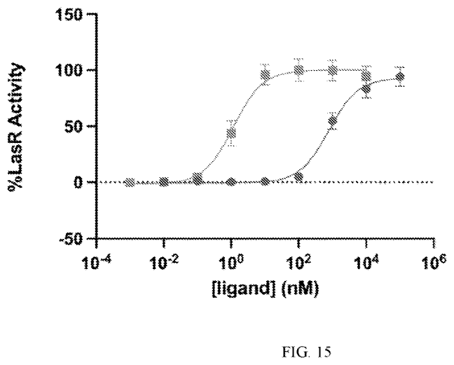

The EC₅₀, 95% Cl, and Max Activation for each sample is reported below. The results of the % LasR Activity (y-axis) and ligand concentration (nM) for each sample are shown in FIGS. 1-15. FIG. 1 shows the results for KMM 5-55-2. FIG. 2 shows the results for KMM 5-56-2. FIG. 3 shows the results for LSD-92. FIG. 4 shows the results for LSD-33. FIG. 5 shows the results for LSD 136. FIG. 6 shows the results for LSD 137. FIG. 7 shows the results for LSD 1-32. FIG. 8 shows the results for LSD 1-34. FIG. 9 shows the results for LSD 1-35. FIG. 10 shows the results for LSD 1-59. FIG. 11 shows the results for LSD 2-47. FIG. 12 shows the results for LSD 2-65. FIG. 13 shows the results for LSD 2-93. FIG. 14 shows the results for LSD 2-113. FIG. 15 shows the results for LSD 2-120.

TABLE 1

| | | | | Maximum |
|---|---|---|---|---|
| | | | | Activation |
| Sample | Structure | EC₅₀ | 95% Cl | (%) |
| KMM 5-55-2 | 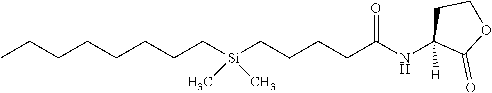 [SMILES: CCCCCCCC[Si](C)(C)CCCCC(=O)N[C@@H]1CCOC1=O] | 45.8 nM | 34.5-60.5 nM | 101 |
| | (S)-5-(dimethyl(octyl)silyl)-N-(2- | | | |
| | oxotetrahydrofuran-3-yl)pentanamide | | | |
| KMM 5-56-2 | 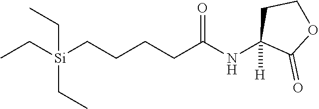 | 22.2 nM | 18-9-26.0 nM | 94.3 |
| | (S)-N-(2-oxotetrahydrofuran-3-yl)-5- | | | |
| | (triethylsilyl)pentanamide | | | |
| LSD-92 | 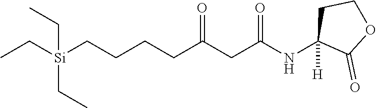 | 113 nM | 92.4-137 nM | 86 |
| | (S)-3-oxo-N-(2-oxotetrahydrofuran-3- | | | |
| | yl)-7-(triethylsilyl)heptanamide | | | |
| LSD 133 (antagonist) | 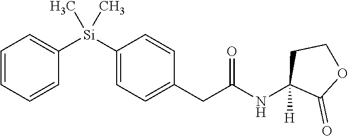 [SMILES: C[Si](C)(c1ccccc1)c1ccc(CC(=O)N[C@@H]2CCOC2=O)cc1] | 15 nM | — | 9 |
| | (S)-2-(4- | | | |
| | (dimethyl(phenyl)silyl)phenyl)-N-(2- | | | |
| | oxotetrahydrofuran-3-yl)acetamide | | | |
| LSD 136 (antagonist) | 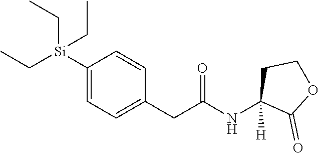 [SMILES: CC[Si](CC)(CC)c1ccc(CC(=O)N[C@@H]2CCOC2=O)cc1] | — | — | 1.4 |
| | (S)-N-(2-oxocyclopentyl)-2-(4- | | | |
| | (triethylsilyl)phenyl)acetamide | | | |
| LSD 137 (antagonist) | 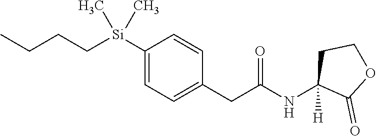 [SMILES: CCCC[Si](C)(C)c1ccc(CC(=O)N[C@@H]2CCOC2=O)cc1] | — | — | 14.1 |
| | (S)-2-(4-(butyldimethylsilyl)phenyl)- | | | |
| | N-(2-oxotetrahydrofuran-3- | | | |
| | yl)acetamide | | | |
| LSD 1-32 | 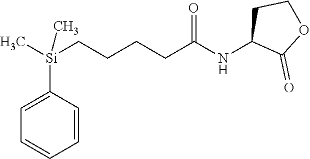 [SMILES: C[Si](C)(CCCCC(=O)N[C@H]1CCOC1=O)c1ccccc1] | 69.22 nM | 59.5-80.4 nM | 96.1 |
| | (S)-5-(dimethyl(phenyl)silyl)-N-(2- | | | |
| | oxotetrahydrofuran-3-yl)pentanamide | | | |
| LSD 1-34 | 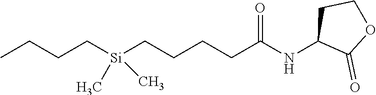 [SMILES: CCCC[Si](C)(C)CCCCC(=O)N[C@H]1CCOC1=O] | 98.3 nM | 86.7-111.5 nM | 101 |
| | (S)-5-(butyldimethylsilyl)-N-(2- | | | |
| | oxotetrahydrofuran-3-yl)pentanamide | | | |
| LSD 1-35 | 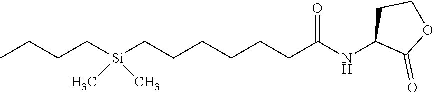 [SMILES: CCCC[Si](C)(C)CCCCCCC(=O)N[C@H]1CCOC1=O] | 218.3 nM | 187.3-254.9 nM | 90.4 |
| | (S)-7-(butyldimethylsilyl)-N-(2- | | | |
| | oxotetrahydrofuran-3-yl)heptanamide | | | |
| LSD 1-59 (inactive) | 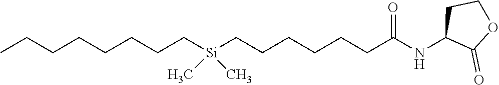 [SMILES: CCCCCCCC[Si](C)(C)CCCCCCC(=O)N[C@H]1CCOC1=O] | — | — | 25 |
| | (S)-7-(dimethyl(octyl)silyl)-N-(2- | | | |
| | oxotetrahydrofuran-3-yl)heptanamide | | | |
| LSD 2-47 | (S)-7-(dimethyl(phenyl)silyl)-3-oxo-N- | 35.5 nM | 30.6-41.2 nM | 99.7 |
| | (2-oxotetrahydrofuran-3- | | | |
| | yl)heptanamide | | | |
| | 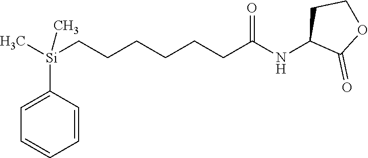 [SMILES: C[Si](C)(CCCCCCC(=O)N[C@H]1CCOC1=O)c1ccccc1] | | | |
| | (S)-7-(dimethyl(phenyl)silyl)-N-(2- | | | |
| | oxotetrahydrofuran-3-yl)heptanamide | | | |
| LSD 2-65 | 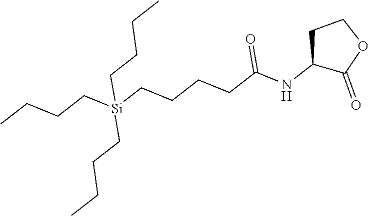 [SMILES: CCCC[Si](CCCC)(CCCC)CCCCC(=O)N[C@H]1CCOC1=O] | 14.4 μM | 10.9-19.1 μM | 79.8 |
| | (S)-N-(2-oxotetrahydrofuran-3-yl)-5- | | | |
| | (tributylsilyl)pentanamide | | | |
| LSD 2-93 | 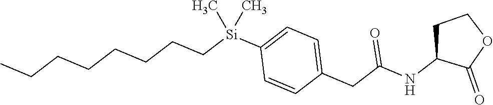 [SMILES: CCCCCCCC[Si](C)(C)c1ccc(CC(=O)N[C@H]2CCOC2=O)cc1] | | | |
| | (S)-2-(4-(dimethyl(octyl)silyl)phenyl)- | | | |
| | N-(2-oxotetrahydrofuran-3- | | | |
| | yl)acetamide | | | |
| LSD 2-113 | 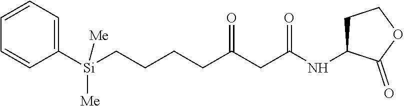 [SMILES: C[Si](C)(CCCCC(=O)CC(=O)N[C@H]1CCOC1=O)c1ccccc1] | 24.7 nM | 20.7-29.4 nM | 106.2 |
| | (S)-7-(dimethyl(phenyl)silyl)-3-oxo-N- | | | |
| | (2-oxotetrahydrofuran-3- | | | |
| | yl)heptanamide | | | |
| LSD 2-120 | 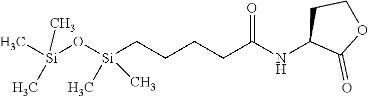 [SMILES: C[Si](C)(C)O[Si](C)(C)CCCCC(=O)N[C@H]1CCOC1=O] | 786 nM | 696-887 nM | 94.0 |
| | (S)-N-(2-oxotetrahydrofuran-3-yl)-5- | | | |
| | (1,1,3,3,3- | | | |
| | pentamethyldisiloxaneyl)pentanamide | | | |

### Example 2

3 compounds were assayed for LasR antagonism. The compounds are shown below, along with IC₅₀, 9500 Cl, and Maximum Activation (0%), if applicable.

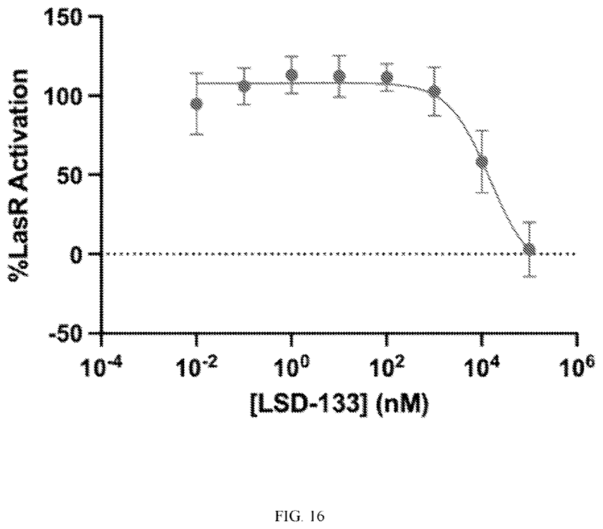

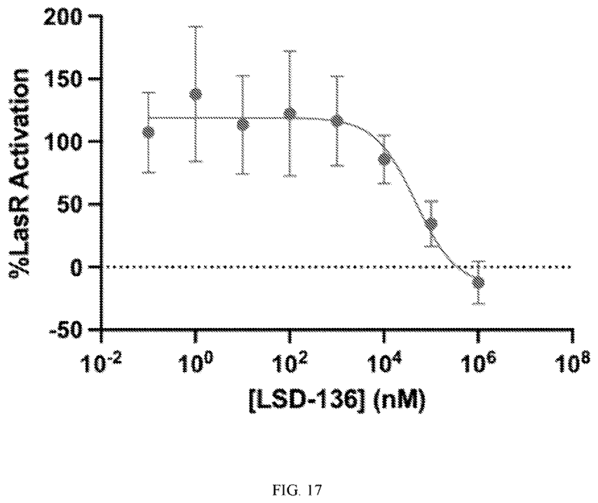

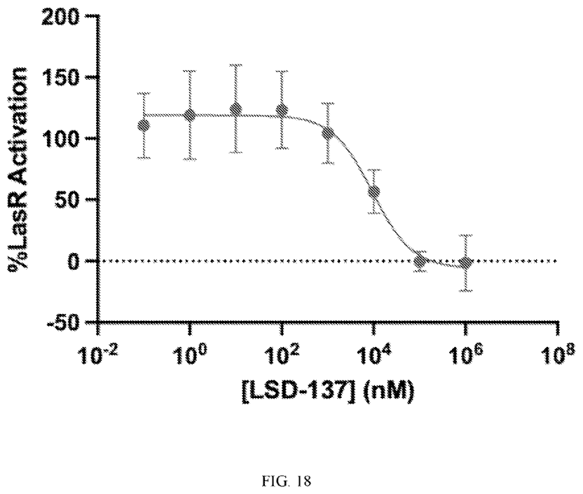

The IC₅₀, 9500 Cl, and Max Activation for each sample is reported below. The results of the 00 LasR Activation (y-axis) and compound concentration (nM) for each sample are shown in FIGS. 16-18. FIG. 16 shows the results for LSD 133. FIG. 17 shows the results for LSD 136. FIG. 18 shows the results for LSD 137.

TABLE 2

| | | | | Maximum |
|---|---|---|---|---|
| | | | | Activation |
| Sample | Structure/Name | IC₅₀ | 95% Cl | (%) |
| LSD 133 | 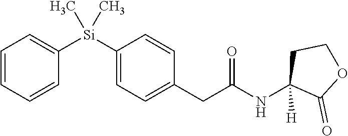 [SMILES: C[Si](C)(c1ccccc1)c1ccc(CC(=O)N[C@@H]2CCOC2=O)cc1] | 14.6 μM | 10.3-21.2 μM | 97.2 |
| | (S)-2-(4- | | | |
| | (dimethyl(phenyl)silyl)phenyl)-N-(2- | | | |
| | oxotetrahydrofuran-3-yl)acetamide | | | |
| LSD 136 | 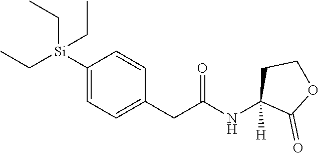 [SMILES: CC[Si](CC)(CC)c1ccc(CC(=O)N[C@@H]2CCOC2=O)cc1] | 46.7 μM | 19.6-100 μM | 112 |
| | (S)-N-(2-oxocyclopentyl)-2-(4- | | | |
| | (triethylsilyl)phenyl)acetamide | | | |
| LSD 137 | 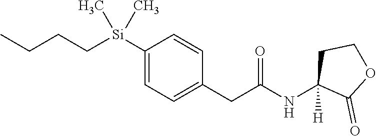 [SMILES: CCCC[Si](C)(C)c1ccc(CC(=O)N[C@@H]2CCOC2=O)cc1] | 9.3 μM | 5.8-14.6 μM | 101 |
| | (S)-2-(4-(butyldimethylsilyl)phenyl)- | | | |
| | N-(2-oxotetrahydrofuran-3- | | | |
| | yl)acetamide | | | |

Based on the results in Tables 1 and 2 and FIGS. 1-18, there was biological activity for several compounds that match activity of known native ligands, or were more active than the nature ligands. The position and structure of the silyl group, as well as the alkyl group substitution and length, contributed to control of the biological activity.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention. All US patents and publications cited herein are incorporated by reference in their entirety.

1. A compound having the formula: 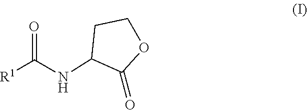
wherein R¹ has the formula: 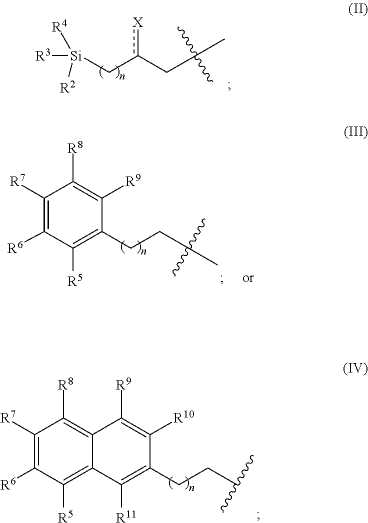
wherein R²; R³, and R⁴ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group;
wherein R⁵, R⁶, R⁷, R⁸, R⁹, R¹⁰, and R¹¹ are each independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group, with the proviso that at least one of R⁵, R⁶, R⁷, R⁸, R⁹, R¹⁰, and R¹¹ comprises Si;
wherein X is hydrogen, or oxygen,
wherein the subscript n is an integer from 0 to 9;
or a pharmaceutically acceptable salt, solvate, or isomer thereof.

2. The compound of claim 1, wherein R¹ is Formula (II).

3. The compound of claim 1, wherein R¹ is Formula (II) and wherein R², R³, and R⁴ are independently selected from a C₁₋₁₀ alkyl group.

4. The compound of claim 1, wherein R¹ is Formula (II) and wherein R², R³, and R⁴ are independently selected from a C₁₋₅ alkyl group.

5. The compound of claim 1, wherein R¹ is Formula (II) and wherein at least two of R², R³, and R⁴ are identical.

6. The compound of claim 1, wherein R¹ is Formula (II) and wherein R², R³, and R⁴ are identical and are selected from a C₁₋₁₀ alkyl group.

7. The compound of claim 1, wherein at least one of R², R³, and R⁴ is a phenyl group or an alkyl phenyl group.

8. The compound of claim 1, wherein R¹ is Formula (III).

9. The compound of claim 1, wherein two of R⁵, R⁶, R⁷, R⁸, and R⁹ are hydrogen.

10-11. (canceled)

12. The compound of claim 1, wherein at least one of R⁵, R⁶, R⁷, R⁸, and R⁹ has the formula: 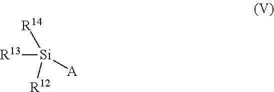
wherein A is at least one of R⁵, R⁶, R⁷, R⁸, and R⁹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

13. The compound of claim 12, wherein at least one of R¹², R¹³, and R¹⁴ is a phenyl group or an alkyl phenyl group.

14. The compound of claim 12, wherein at least one of R¹², R¹³, and R¹⁴ is an alkyl group.

15. The compound of claim 1, wherein R¹ is Formula (IV).

16. The compound of claim 1, wherein two of R⁵, R⁶, R⁷, R⁸, R⁹, R¹⁰ and R¹¹ are hydrogen.

17-20. (canceled)

21. The compound of claim 1, wherein at least one of R⁵, R⁶, R⁷, R⁸, R⁹, R¹⁰ and R¹¹ has the formula: 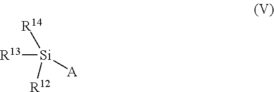
wherein A is at least one of R⁵, R⁶, R⁷, R⁸, R⁹, R¹⁰ and R¹¹;
wherein R¹², R¹³, and R¹⁴ are independently selected from the group consisting of hydrogen, C₁₋₁₀ alkyl, C₂₋₁₀ alkenyl, a phenyl group, an alkyl phenyl group, a naphthyl group, an alkyl naphthyl group, a silyl group and a siloxy group.

22. The compound of claim 21, wherein at least one of R¹², R¹³, and R¹⁴ is a phenyl group.

23. The compound of claim 21, wherein at least one of R¹², R¹³, and R¹⁴ is an alkyl group.

24. The compound of claim 1, wherein X is O.

25. The compound of claim 1, wherein X is H.

26. The compound of claim 1, wherein the compound is:
(S)—N-(2-oxotetrahydrofuran-3-yl)-5-(triethylsilyl)pentanamide,
(S)-5-(dimethyl(octyl)silyl)-N-(2-oxotetrahydrofuran-3-yl)pentanamide,
(S)-2-(4-(butyldimethylsilyl)phenyl)-N-(2-oxotetrahydrofuran-3-yl)acetamide,
(S)-3-oxo-N-(2-oxotetrahydrofuran-3-yl)-7-(triethylsilyl)heptanamide,
(S)-2-(4-(dimethyl(phenyl)silyl)phenyl)-N-(2-oxotetrahydrofuran-3-yl)acetamide,
(S)—N-(2-oxocyclopentyl)-2-(4-(triethylsilyl)phenyl)acetamide,
(S)-5-(dimethyl(phenyl)silyl)-N-(2-oxotetrahydrofuran-3-yl)pentanamide,
(S)-5-(butyldimethylsilyl)-N-(2-oxotetrahydrofuran-3-yl)pentanamide,
(S)—N-(2-oxotetrahydrofuran-3-yl)-5-(tributylsilyl)pentanamide,
(S)—N-(2-oxotetrahydrofuran-3-yl)-5-(1,1,3,3,3-pentamethyldisiloxaneyl)pentanamide,
(S)-7-(dimethyl(phenyl)silyl)-N-(2-oxotetrahydrofuran-3-yl)heptanamide,
(S)-7-(butyldimethylsilyl)-N-(2-oxotetrahydrofuran-3-yl)heptanamide,
(S)—N-(2-oxotetrahydrofuran-3-yl)-7-(triethylsilyl)heptanamide,
(S)-7-(dimethyl(phenyl)silyl)-3-oxo-N-(2-oxotetrahydrofuran-3-yl)heptanamid,
(S)-7-(butyldimethylsilyl)-3-oxo-N-(2-oxotetrahydrofuran-3-yl)heptanamide,
(S)-2-(4-(dimethyl(octyl)silyl)phenyl)-N-(2-oxotetrahydrofuran-3-yl)acetamide, or
(S)-7-(dimethyl(octyl)silyl)-N-(2-oxotetrahydrofuran-3-yl)heptanamide.

27-43. (canceled)
